(19) United States
(12) Patent Application Publication
(10) Pub. No.: US 2014/0256015 A1
(43) Pub. Date: Sep. 11, 2014

(54) CAGED COMPOUND WHICH CAN BE PHOTOACTIVATED SELECTIVELY FOR CELL TYPES

(71) Applicant: Toho University, Tokyo (JP)

(72) Inventors: Toshiaki FURUTA, Funabashi-shi (JP); Masaki SONE, Funabashi-shi (JP); Akinobu SUZUKI, Funabashi-shi (JP); Eri OKA, Ichikawa-shi (JP)

(73) Assignee: Toho University, Tokyo (JP)

(21) Appl. No.: 14/189,629

(22) Filed: Feb. 25, 2014

(30) Foreign Application Priority Data
Feb. 26, 2013 (JP) 2013-036379
Feb. 21, 2014 (JP) 2014-032218

(51) Int. Cl. A61K 41/00; A61K 31/7076; A61K 31/337; G01N 33/58

(52) U.S. Cl. CPC A61K 41/0042; G01N 33/582; A61K 31/7076; A61K 31/337
USPC 435/173.1; 536/17.9; 536/18.1; 536/17.1

(57) ABSTRACT

An objective of the present invention is to provide a caged compound that can be photoactivated selectively for specific target cell types and can be used in an individual organism. The objective can be achieved by a compound represented by general formula K-Q-X, which is prepared by binding bioactive substance X, photocleavable protecting group Q, and compound K, which can be an enzyme substrate and is dissociated from Q-X by an enzyme reaction, wherein: Q is a protecting group that is photocleaved by light with a specific wavelength and then dissociated from X, when K is not bound thereto; X is a substance that does not express bioactivity when Q is bound thereto, but expresses bioactivity when Q is dissociated therefrom; and K is dissociated from Q by the above enzyme, so as to form a compound represented by Q-X. Specifically, the objective can be achieved with the use of such a compound, which is characterized in that Q is dissociated from X when the compound represented by Q-X is photoirradiated, and thus K expresses bioactivity.

### BACKGROUND OF THE INVENTION

1. Field of the Invention

The present invention relates to a caged compound exhibiting bioactive effects within specific target cells through photoactivation.

2. Background Art

The only known method to control cellular physiological functions under conditions reproduced as physiologically accurately as possible, especially with a high spatiotemporal resolution, is photocontrol using a high-resolution microscope. Furthermore, the use of an appropriately designed caged compound has been essential to perform photocontrol. The term “caged compound” refers to a bioactive molecule, into which a photocleavable protecting group has been introduced, the activity of which can be turned on (or off) by photoirradiation. Caged compounds with excellent properties have been developed (e.g., ACS Chem. Biol. 2009, 4, 409). As a result, it has become possible to achieve photostimulation with a very high spatial resolution in cultured cells, brain slices, or fixed and immobilized individual subjects by introducing a caged compound such as a neurotransmitter or an intracellular second messenger, and then focusing excitation light using a microscopic lens.

For example, it has been demonstrated that local stimulation, by which only a portion within a cell is stimulated, can be effectively applied to studies for the elucidation of the detailed mechanism of intracellular signal transduction when T cells recognize antigen-presenting cells (Nature Immun. 2009, 10, 627.). It has also been demonstrated that a specific intracellular signal transduction pathway can be instantly activated by photoirradiation without providing any perturbation to sperm cell motility. Moreover, the spatiotemporal dynamics of calcium ions can be monitored in real time using a fluorescent sensor in combination with caged compounds (e.g., ChemBioChem, 2004, 5, 1119 and J. Cell Biol. 2005, 169, 725). However, the number of examples of the use of existing caged compounds for individual organisms is limited (e.g., Nature Genet. 2001, 28, 317; Cell 2005, 121, 141; and Biotechniques, 2007, 43, 161).

The major reasons for this are the impossibility of using photoirradiation to target specific cells of a moving individual, and the fact that the optimum wavelength to be used for photostimulation is present in the ultraviolet range, making it difficult for light to reach the deep interior from the body surface.

Meanwhile, in the brain•neuroscience research fields, the use of channel rhodopsins has resulted in a huge breakthrough in the photocontrol of individual behavior (e.g., Annu. Rev. Neurosci. 2011, 34, 389). A transgenic individual expressing channel rhodopsin in specific neurons can be produced using the advantage of being encoded by a gene. The application thereof to an experiment obviates the need for targeting that uses photoirradiation itself. Light is emitted over a relatively wide range, but specific neurons can be stimulated with light. An analysis of the activity of an individual neuron in association with individual behavior is becoming possible. However, under current circumstances, the physiological functions that can be photocontrolled are almost limited to neuronal excitation and suppression. Furthermore, artificiality and abnormality can also be problematic, because all of the targeted neurons are altered to be photosensitive from birth. Therefore, there is a strong need to realize the photocontrol of various physiological functions so as to develop a caged compound designed to possess both the advantage of channel rhodopsin as a protein capable of targeting a specific cell and the advantage of a caged compound as a small molecule organic compound that can be added to a normal individual after birth in such a way that it will function only within the target cells of the relevant individual.

### SUMMARY OF THE INVENTION

### Objective to be Attained by the Invention

The use of an existing caged compound within an individual organism is difficult. The major reasons for this are as follows: using photoirradiation to target specific cells of a moving individual is difficult, making it impossible to realize high spatial resolution (problem 1), and the optimum wavelength for photostimulation is in the ultraviolet range, making it difficult for light to reach the deep interior from the body surface (problem 2).

An objective of the present invention is to address these problems so as to provide a caged compound that can be selectively photoactivated for specific target cell types and thus can be used in an individual organism.

### Means for Attaining the Objective

The present inventors have added new functions to the Bhc-photocleavable protecting group thought to have the best performance at present (e.g., Proc. Natl. Acad. Sci. U.S.A., 1999, 96, 1193; Trends Anal. Chem. 2004, 23, 501; J. Synth. Org. Chem. Jpn., 2012, 69, 1164) and thus have developed and examined a novel caged compound that can distinguish a cell type from other cell types and can be photoactivated only within or in the vicinity of the target cells, in order to address problem 1. To address problem 2, the present inventors have examined the development of a novel photocleavable protecting group that can be photodeprotected using visible light.

The present inventors have discovered that problem 1 can be addressed by a new caged compound. This new caged compound is developed by further protecting a photocleavable protecting group of an existing caged compound with a substance, which is a substrate of a specific enzyme and can be cleaved with the enzyme, wherein the photocleavable protecting group is not photoactivated when it is protected with the substrate binding thereto, but the photocleavable protecting group can be photoactivated when the substrate is cleaved with the specific enzyme so as to be dissociated from the photocleavable protecting group. The present inventors have further discovered that problem 2 can be addressed by the photocleavable protecting group that can be photoactivated by light with a wavelength higher than 500 nm. Thus, the present inventors have completed the present invention.

Specifically, the present invention is as follows.

[1] A compound represented by the following general formula (I), in which bioactive substance X, photocleavable protecting group Q, and compound K, which can be an enzyme substrate and is dissociated from Q-X by an enzyme reaction are bound.

[Chemical formula 1]

K-Q-X (I)

[2] The compound according to [1] represented by the following general formula (I), in which the bioactive substance X, the photocleavable protecting group Q, and the compound K, which can be an enzyme substrate and is dissociated from Q-X by an enzyme reaction ae bound, wherein

Q is a protecting group that is photocleaved by light with a specific wavelength and then dissociated from X, when K is not bound thereto;

X is a substance that does not express bioactivity when Q is bound thereto, but expresses bioactivity when Q is dissociated therefrom; and

K is dissociated from Q by the enzyme so as to generate a compound represented by Q-X, and K expresses bioactivity when the compound represented by Q-X is photoirradiated to cause the dissociation of Q from X.

[Chemical formula 2]

K-Q-X (I)

[3] The compound according to [1] or [2], wherein the enzyme for causing the dissociation of the compound K, which can be an enzyme substrate, from Q-X, is an intracellular endogenous enzyme or an exogenous enzyme that is introduced into cells, and X expresses bioactivity within cells.

[4] The compound according to any one of [1] to [3], wherein the photocleavable protecting group Q is selected from the group consisting of a Bhc group, a Bhcmoc group, a xanthene-type photocleavable protecting group, and a resorufin-type photocleavable protecting group.

[5] The compound according to any one of [1] to [4], wherein a combination of the compound that can be an enzyme substrate and the enzyme is selected from the group consisting of a combination of galactose and β-galactosidase, a combination of glucose and glucosidase, a combination of glucuronic acid and glucuronidase, and a combination of phosphoric acid and alkaline phosphatase.

[6] The compound according to any one of [1] to [5], wherein the bioactive substance X is selected from the group consisting of a protein, a nucleic acid, a fatty acid, and an amino acid.

[7] The compound according to any one of [1] to [5], wherein the bioactive substance X is selected from the group consisting of an enzyme inhibitor, a hormone, a lipid-signal molecule, a neurotransmitter, a microtubule depolymerization inhibitor, a microtubule polymerization inhibitor, an antitumor antibiotic, a topoisomerase inhibitor, a purine metabolism inhibitor, a ribonucleotide reductase inhibitor, a pyrimidine metabolism inhibitor, an antifolate, and an alkylating agent.

[8] The compound according to any one of [1] to [7], wherein the photocleavable protecting group Q is a Bhc group, the compound K, which is an enzyme substrate and is dissociated from Q-X by an enzyme reaction, is galactose, and galactose is dissociated from Q-X by β-galactosidase.

A method for causing X to express bioactivity within cells, comprising the steps of: introducing the compound K-Q-X of any one of [1] to [8] into cells; causing the dissociation from Q-X of the compound K, which can be an enzyme substrate and is dissociated from Q-X by an enzyme reaction within cells; and causing the dissociation of Q from Q-X by photoirradiation.

[10] The method for causing X to express bioactivity within cells according to [9], wherein the enzyme is a cell endogenous enzyme.

[11] The method for causing X to express bioactivity within cells according to [9], wherein the enzyme is an exogenous enzyme.

[12] The method for causing X to express bioactivity within cells according to any one of [9] to [11], wherein the photocleavable protecting group Q is a Bhc group or a Bhcmoc group, and photoirradiation is performed with a wavelength ranging from 400 nm to 450 nm.

[13] The method for causing X to express bioactivity within cells according to any one of [9] to [12], wherein the compound K, which is an enzyme substrate and is dissociated from Q-X by an enzyme reaction, is galactose, and galactose is dissociated from Q-X by β-galactosidase.

[14] A precursor compound of the compound represented by K-Q-X according to any one of [1] to [8], which:

is represented by K-Q, wherein the photocleavable protecting group Q and the compound K, which can be an enzyme substrate and is dissociated from Q-X by an enzyme reaction, are bound to each other; and

is converted to K-Q-X by binding the bioactive substance X thereto.

[15] The precursor compound according to [14], wherein:

the bioactive substance X that can be bound to the precursor compound is a compound selected from the group consisting of DNA, cDNA, mRNA, siRNA, miRNA, antisense RNA, ATP, ADP, cAMP, GTP, GDP, GTP-γ-A, GDP-β-S, cGMP, 8-bromo cAMP, 8-chloro cAMP, 8-bromo cGMP, 8-chloro cGMP, 8-para-chlorophenylthio cAMP, and 8-para-chlorophenylthio cGMP;

the photocleavable protecting group Q is a Bhc group or a Bhcmoc group to which a diazo group is bound; and

the compound K, which can be an enzyme substrate, is galactose.

### Effect of the Invention

Whereas a conventional existing caged compound is characterized by the masking of the activity of a bioactive substance using a key referred to as a photocleavable protecting group, the caged compound of the present invention is characterized by the masking of the photoreactivity of a photocleavable protecting group using another key referred to as an enzyme substrate. In the present invention, the enzyme substrate is referred to as the “first key,” and the photocleavable protecting group is referred to as the “second key.” As a result, the caged compound of the present invention is characterized by the fact that a substrate is dissociated by an enzyme reaction only at the site(s) of an enzyme capable of digesting the substrate contained in the caged compound, thereby opening the first key. When the first key is opened, the photoreactivity of the photocleavable protecting group used as the second key is restored. The photocleavable protecting group is dissociated because of the light, and the second key is opened to finally cause the bioactive substance to express its activity. Cells containing an endogenous or exogenous specific enzyme for a substrate functioning as the first key are targeted, so that the second key is opened only within or in the vicinity of the target cells. Photoirradiation is further performed, so that the second key is opened. As a result, the bioactive substance can be caused to express activity only within specific target cells.

Through the use of the caged compound of the present invention, which can be selectively photoactivated for cell types, a bioactive substance can be caused to express activity only within the specific target cells. For example, when a bioactive substance is a therapeutic agent such as an anticancer agent, setting a first key that is opened only within the cancer cells makes it possible to activate the anticancer agent only within the cancer cells.

### BRIEF DESCRIPTION OF THE DRAWINGS

FIG. 1 shows the structure of a Bhc group.

FIG. 2 shows the structure of a Bhcmoc group.

FIG. 3 shows the structures of a xanthene-type photocleavable protecting group bound to galactose and a resorufin-type photocleavable protecting group bound to galactose.

FIG. 4 shows the combinations of enzymes and compounds that can be enzyme substrates.

FIG. 5 shows the structure of Gal-Bhc-X, enzyme reaction, and photoreaction.

FIG. 6 shows examples of the caged compound of the present invention.

FIG. 7 shows the UV spectra of Bhcmoc-propargyl and Gal-Bhcmoc-propargyl.

FIG. 8 shows decreases in Bhcmoc-propargyl and Gal-Bhcmoc-propargyl due to photocleaving.

### DETAILED DESCRIPTION OF THE PREFERRED EMBODIMENTS

Hereafter, the present invention is described in detail.

The present invention relates to a caged compound that acquires the capacity of being photoactivated only within specific target cells and thus can be photoactivated selectively for cell types.

### 1. Structure of the Caged Compound of the Present Invention

The compound of the present invention is represented by the following general formula (I)

[Chemical formula 3]

K-Q-X (I)

X is a bioactive substance, Q is a photocleavable protecting group, and

K is a compound, which can be an enzyme substrate and is dissociated from Q-X by an enzyme reaction. Here, the expression, “a compound, which can be an enzyme substrate and is dissociated from Q-X by an enzyme reaction” refers to a compound such that in compound K-Q-X, a K-Q linkage is degraded by hydrolysis or the like as a result of an enzyme reaction, and thus K can be dissociated from Q-X. For example, when K is galactose, a hydroxy group of galactose and a photocleavable protecting group are bound via an ether linkage, so as to form β-galactoside. β-galactosidase generates galactose by hydrolysis with β-galactoside as a substrate. However, in the present invention, galactose is referred to as a compound that can be a substrate of β-galactosidase.

X alone is a bioactive substance having bioactive effects within cells. When X binds to Q, and its functional group is protected with a photocleavable protecting group, its activity is masked, and X does not express bioactivity.

A photocleavable protecting group of a caged compound is photoactivated by irradiation with a specific wavelength, and a photocleavable reaction occurs. The photocleavable protecting group is dissociated from K as a result of deprotection, and thus K expresses its bioactive effects.

However, when K is bound to Q-X, Q is not photoactivated even when photoirradiated with a wavelength of more than 400 nm and is not dissociated from K. Hence, X does not express bioactive effects.

When K is first dissociated from compound K-Q-X by an enzyme reaction, a caged compound represented by Q-X, in which bioactive substance X is binding to a photocleavable protecting group, is generated. When the caged compound is photoirradiated, photocleavable protecting group Q is dissociated from Q-X to cause K to express bioactivity.

Specifically, K-Q-X is introduced into specific cells to cause an enzyme reaction to take place within the cells, and thus K is dissociated from K-Q-X. Next, the Q-X remaining within the cells is photoirradiated, so that the photocleavable protecting group Q is dissociated to cause X to express bioactivity. Specifically, in a caged compound wherein a bioactive substance is protected using a photocleavable protecting group, the photosensitivity of the photocleavable protecting group is masked by compound K, which can be an enzyme substrate. In the present invention, masking the photosensitivity of a photocleavable protecting group using a compound that can be an enzyme substrate is referred to as the first key, and inhibiting the expression of the bioactive effects of a bioactive substance using a photocleavable protecting group is referred to as the second key.

A bioactive substance represented by X is not limited and any bioactive substance can be used herein, as long as it is a substance having a functional group that can be protected with a photocleavable protecting group and loses bioactivity or exhibits at least partially suppressed bioactivity, when protected with a photocleavable protecting group via the functional group. Here, the term “bioactive substance” refers to a substance or a compound that exhibits physiological action or pharmacologic action on an organism. Examples of such a bioactive substance include biomaterials and synthetic compounds. Moreover, in the present invention, a compound that is a complex of a fluorescent dye or the like and a compound that can bind to a substance within a cell in vivo emits signals in vivo, and thus can be used for detection of a substance or the like in vivo, is also referred to as a “bioactive substance” in view of its reactivity with a substance in vivo.

Examples of a functional group that can be protected with a photocleavable protecting group include carboxylic acid, phosphoric acid, sulfonic acid, amide, primary or secondary amine, alcohol, phenol, thiol, ketone, and aldehyde. Bioactive substances having these functional groups can be used as bioactive substances for the caged compound of the present invention.

Examples of such bioactive substances include cytokines (e.g., various interleukins, interferons, tumor necrosis factors, and growth factors), proteins and peptides such as antibodies; amino acids such as glutamic acid, and cysteine; nucleic acids such as DNA, cDNA, mRNA, siRNA, miRNA, and antisense RNA; sugars, oligosaccharide, and polysaccharide such as glucose, galactose, mannose, fructose, and lactose; fatty acids such as arachidonic acid, linoleic acid, linolenic acid, eicosapentaenoic acid, and docosahexaenoic acid; lipids; nucleotides such as ATP, ADP, cAMP, GTP, GDP, GTP-γ-A, GDP-β-S, cGMP, 8-bromo cAMP, 8-chloro cAMP, 8-bromo cGMP, 8-chloro cGMP, 8-para-chlorophenylthio cAMP, and 8-para-chlorophenylthio cGMP; phosphoric acids such as phosphoric acid and phosphoric acid ester; chelating agents such as EDTA and EGTA; ionophores such as nigrosine; nucleosides such as cADP ribose, 8-amino-cADP-ribose, and 8-bromo-cADP-ribose; cyclitol such as inositol and inositol phosphates such as myo-inositol phosphate, myo-inositol-1,4,5-triphosphate, myo-inositol-1,3,4,5-tetrakisphosphate, and myo-inositol-3,4,5,6-tetrakisphosphate; fluorescent dyes such as fluorescein, fluorescent substances such as rhodamine and fluorescent protein or a complex of such a fluorescent substance and a compound binding to a substance existing within cells; and low molecular weight compounds. Furthermore, when bioactive substances are classified based on the functions, examples thereof include various hormones such as insulin, glucagon, thyroid-stimulating hormone, epinephrine, estrogen, and progesterone; various enzyme inhibitors; lipid-signal molecules such as diacylglycerol; antibiotics such as penicillin; and neurotransmitters such as glutamic acid, aspartic acid, carbamylcholine, dopamine, epinephrine, GABA, glycine, haloperidol, isoproterenol, kainic acid, MMDA, NMDA receptor antagonist MK-801, norepinephrine, phenylephrine, propranolol, and serotonin. Moreover, various medicines composed of macromolecular compounds or low molecular weight compounds can also be used as bioactive substances. Examples of such medicines include various anticancer agents. Examples of such an anticancer agent include microtubule depolymerization inhibitors such as paclitaxel (Taxol) and docetaxel (Taxotere); antitumor antibiotics such as doxorubicin, mitomycinC, bleomycin, epirubicin, daunorubicin, and bleomycin; microtubule polymerization inhibitors such as vinblastine, vincristine, and vindesine; topoisomerase inhibitors such as irinotecan and nogitecan; purine metabolism inhibitors such as azathioprine and pentostatin; ribonucleotide reductase inhibitors such as hydroxyurea; pyrimidine metabolism inhibitors such as flucytosine; antifolates such as sulfadiazine, sulfamethoxazole, methotrexate, trimethoprim, and pyrimethamine; and alkylating agents such as cyclophosphamide, ifosfamide, melphalan, busulfan, nimustine, ranimustine, dacarbazine, procarbazine, temozolomide, and bendamustine.

These bioactive substances can be bound to photocleavable protecting groups using functional groups such as carboxylic acid, phosphoric acid, sulfonic acid, amide, primary or secondary amine, alcohol, phenol, thiol, ketone, and aldehyde. For example, a protein, a peptide, an amino acid, or the like has a functional group such as carboxylic acid, amide, amine, alcohol, and thiol. A nucleic acid such as DNA and RNA has a functional group such as amide, amine, and phosphoric acid. These functional groups are used for binding to photocleavable protecting groups, so as to protect with the photocleavable protecting groups.

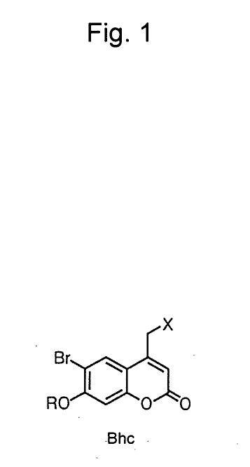

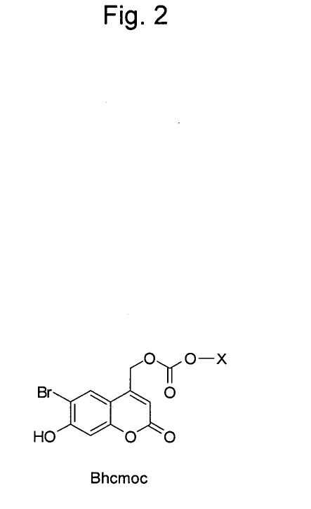

An example of a photocleavable protecting group represented by Q is a photocleavable protecting group that is degraded by irradiation with light having a specific wavelength, so as to be dissociated from caged compound Q-X, as well as binds to compound K, which can be an enzyme substrate, so as to lock the photosensitivity. An example of such a group is a (6-bromo-7-hydroxycoumarin-4-yl)methyl group (Bhc group). The Bhc group has the structure shown in FIG. 1. The bioactive substance X may be bound to the portion thereof represented by X. Furthermore, a (6-bromo-7-hydroxycoumarin-4-yl)methoxycarbonyl group (Bhcmoc group) having the structure shown in FIG. 2 may also be used herein. Moreover, 6-bromo-4-diazomethyl-7-hydroxycoumarin (Bhc-diazo) that is a diazomethane derivative of Bhc may be used as a nucleic acid-related substance such as a nucleic acid, a nucleotide, and a nucleoside.

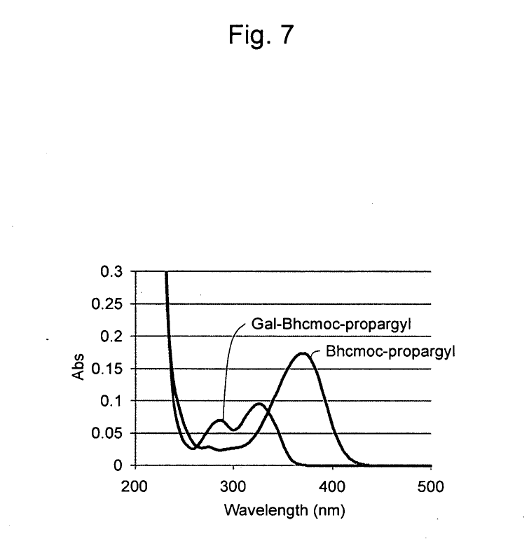

The Bhc group has a phenolic hydroxy group at position 7, which is ionized at pH7, and has an absorption maximum at 370 nm under a physiological environment such as a near-neutral intracellular environment (e.g., J. Synth. Org. Chem. Jpn., 2012, 69, 1164). Meanwhile, in the case of a Bhc group derivative in which the position has been alkylated, the absorption maximum shifts close to 330 nm regardless of the pH of the aqueous solution (Org. Lett. 2007, 9, 4717). Accordingly, the absorption maximum of a caged compound, wherein compound K, which can be an enzyme substrate, is bound at position 7, shifts to a wavelength shorter than 370 nm. For example, photoactivation cannot be performed with visible light (e.g., 405 nm). However, when compound K, which can be a substrate, is dissociated within enzyme-expressing cells, so as to be converted to a Bhc group, a photocleavable protecting group is photoactivated with light with 405 nm, and thus the photocleavable protecting group can be dissociated from the bioactive substance (FIG. 7).

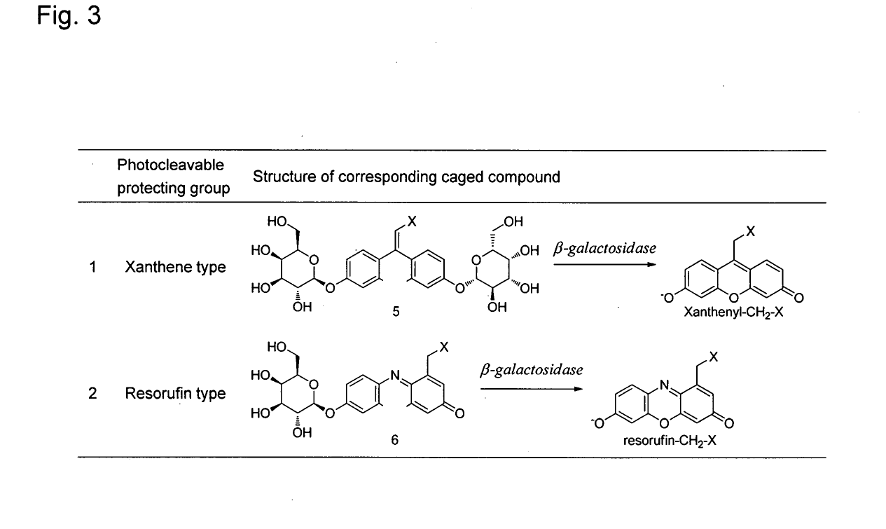

Furthermore, a xanthene-type photocleavable protecting group or a resorufin-type photocleavable protecting group can also be used. FIG. 3 shows the xanthene-type photocleavable protecting group bound to galactose and the resorufin-type photocleavable protecting group bound to galactose. FIG. 3 also shows the structures of caged compounds corresponding to the photocleavable protecting groups. An example of a caged compound of the xanthene-type photocleavable protecting group and X is xanthenyl-CH₂—X. The xanthene-type photocleavable protecting group or the resorufin-type photocleavable protecting group is photoactivated and photocleaved with light having a wavelength longer than that of the Bhc group. For example, the xanthene-type photocleavable protecting group has an absorption maximum at 516 nm.

A compound represented by K, which can be an enzyme substrate, is a compound that can be a substrate of an intracellular endogenous enzyme or an exogenous enzyme (that is introduced from outside). Examples of a combination of the compound K, which can be an enzyme substrate, and the enzyme include a combination of galactose and β-galactosidase, a combination of glucose and glucosidase, a combination of glucuronic acid and glucuronidase, and a combination of phosphoric acid and alkaline phosphatase.

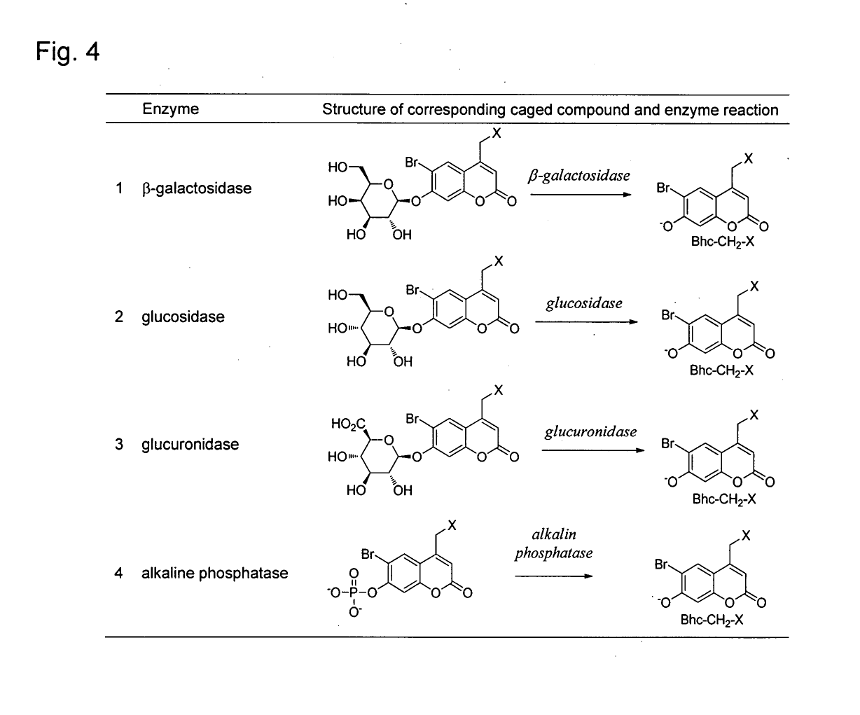

Galactose binds to photocleavable protecting group Q to form β-galactoside, and β-galactosidase hydrolyzes β-galactoside, so that galactose is dissociated from Q-X. Glucose binds to the photocleavable protecting group Q via a glycosidic linkage. Glucosidase hydrolyzes the glycosidic linkage, and thus glucose is dissociated from Q-X. Glucuronic acid binds to the photocleavable protecting group Q via a glucuronide linkage. Glucuronidase hydrolyzes the glucuronide linkage, and then glucuronic acid is dissociated from Q-X. Phosphoric acid binds to the photocleavable protecting group Q via a phosphoric ester linkage. Alkaline phosphatase hydrolyzes the phosphoric ester linkage, and then phosphoric acid is dissociated from Q-X. FIG. 4 shows the structure of the caged compound of the present invention, wherein galactose, glucose, glucuronic acid, or phosphoric acid binds to conjugate Q-X of a photocleavable protecting group and a bioactive substance, and the dissociation reaction of compound K (which can be an enzyme substrate) from Q-X by the enzyme, that is, from the caged compound.

Moreover, as a compound that can be an enzyme substrate, a protein or a peptide having a specific amino acid sequence and protease having high substrate specificity, which is capable of recognizing the specific amino acid sequence and cleaving the protein or the peptide at the relevant site can also be used in combination. For example, trypsin hydrolyzes a peptide linkage on the side of the carboxyl group of a basic amino acid (lysine or arginine). A protein or a peptide is bound to a photocleavable protecting group, so that an amino acid sequence to be cleaved by protease is present at the portion for binding with the photocleavable protecting group. The protein or the peptide binding to the photocleavable protecting group can then be dissociated therefrom using protease.

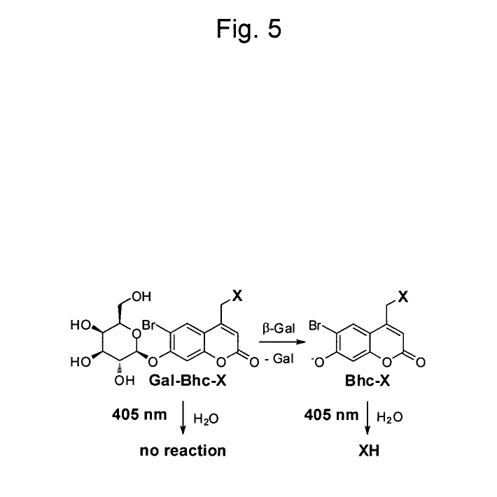

From among the above combinations, a compound represented by Gal-Bhc-X wherein compound K, which can be an enzyme substrate, is galactose (Gal) and photocleavable protecting group Q is a Bhc group can be used as the caged compound of the present invention. FIG. 5 shows the structure of Gal-Bhc-X, an enzyme reaction, and a photoreaction. As shown in FIG. 5, Gal-Bhc-X; that is, in a state in which the first key, Gal, is binding to Bhc-X, the photocleavable protecting group does not undergo photoreaction even when irradiated with light having a wavelength of 405 nm. However, when β-galactosidase (β-Gal) is caused to act thereon, Gal is dissociated from Gal-Bhc-X as a result of an enzymatic reaction, thereby generating a compound represented by Bhc-X. When the compound is irradiated with light having a wavelength of 405 nm, the photocleavable protecting group is deprotected and dissociated from X as a result of a photoreaction, and thus bioactive substance X expresses activity.

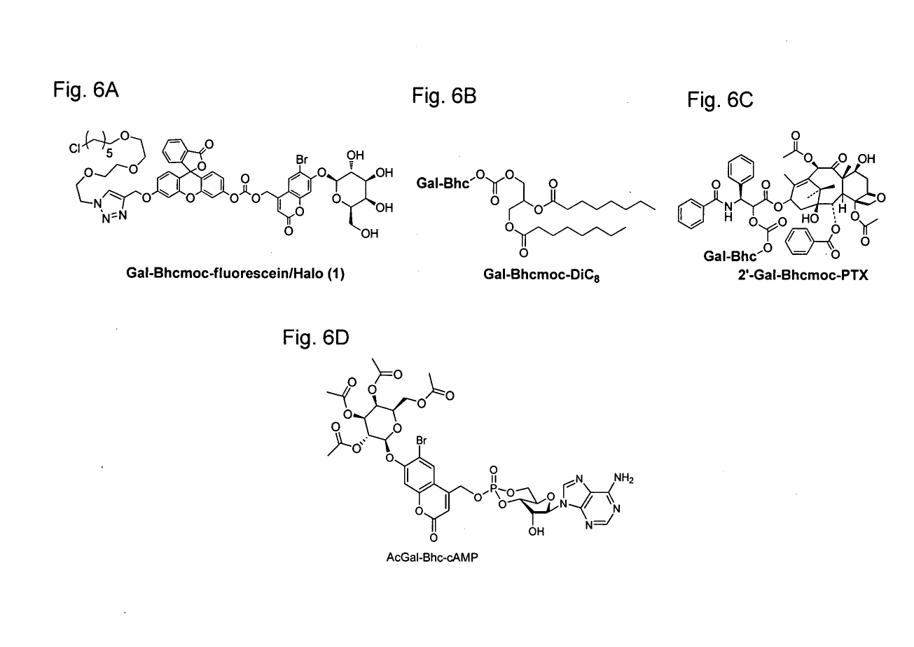

Furthermore, FIG. 6 shows an example of the caged compound of the present invention represented by K-Q-X. FIG. 6A shows the structure of Gal-Bhcmoc-fluorescein wherein fluorescein was used as a bioactive substance.

FIG. 6B shows the structure of Gal-Bhcmoc-DiC₈ wherein diacylglycerol (DiC₈) was used as a bioactive substance. Moreover, FIG. 6C shows the structure of Gal-Bhcmoc-PTX wherein paclitaxel (PTX) (anticancer agent) was used as a bioactive substance. FIG. 6D shows the structure of AcGal-Bhc-cAMP wherein cAMP was used as a bioactive substance.

A compound that can be an enzyme substrate may be bound to a Bhc group as a photocleavable protecting group or a phenolic OH group that is a xanthene-type or a resorufin-type photocleavable protecting group.

To cause dissociation of K from the compound K-Q-X of the present invention within cells, an enzyme capable of cleaving with the use of K as a substrate within cells is caused to act. As such an enzyme, cells' original endogenous enzyme, or an exogenous enzyme (which is not a cells' original enzyme) within cells can be used.

In the present invention, examples of cells include cultured cells and in vivo cells. The caged compound of the present invention can be applied in vitro to cultured cells or in vivo cells isolated, or the caged compound of the present invention can also be applied in vivo to cells within an individual organism. Examples of cells include cells of microorganisms such as bacteria and yeast, and cells of all tissues and organs of plants, insects, birds, non-human animals, and humans.

An exogenous enzyme can be introduced into cells by introducing DNA encoding the enzyme protein into cells, and then causing the expression thereof within cells, for example. A gene can be introduced into cells by a known method. For example, DNA encoding an enzyme protein is inserted into a vector or a plasmid, and then cells can be transformed with the vector or the plasmid. Target cells are infected with a virus (e.g., a viral vector such as adenovirus, adeno-associated virus, and retrovirus vectors) into which DNA has been inserted. A target gene can also be introduced into cells or tissues using a recombinant expression vector constructed via incorporation of the target gene into a gene expression vector (e.g., a plasmid vector) without using viruses. For example, a gene can be introduced into cells by a lipofection method, a phosphoric acid-calcium coprecipitation method, a DEAE-dextran method, a direct DNA injection technique using a micro glass tube, or the like. Moreover, a vector, in which DNA has been inserted, can be incorporated into cells by a gene transfer technique using an internal liposome, a gene transfer technique using an electrostatic type liposome, an HVJ-liposome method, a modified HVJ-liposome method (HVJ-AVE liposome method), a method using an HVJ-E (envelope) vector, a receptor-mediated gene transfer technique, a method for transferring a DNA molecule together with carriers (metal particles) using a particle gun into cells, a direct naked-DNA transfer technique, a gene transfer technique using various polymers, or the like.

At this time, when cells into which the caged compound of the present invention is introduced to cause bioactive substance X to act are specific cells, an enzyme is introduced into only the specific cells. A known compound delivery system can be employed for this. For example, a compound that specifically binds to a protein or a sugar that is expressed on the structures of such specific cells is bound to the surface of a vector in which DNA encoding an enzyme has been introduced. For example, an antibody against a protein or a sugar that is expressed on the cell surface can be used herein. When target cells are cancer cells, antibodies against cancer-specific antigens can be used, for example. The thus constructed vectors are accumulated and bind to target specific cells, and are then incorporated into the cells, so that the enzyme is expressed within the cells. In addition, the vectors can bind to cells so that the enzyme can be expressed around the cells. For example, cancer cells express integrin at high levels. Hence, an RGD (arginine-glycine-aspartic acid) peptide is bound to the surface of a vector, so that cancer cells can be targeted. As described above, an enzyme that cleaves K targeting the caged compound of the present application is introduced in a cell-type-specific manner, so as to be able to cause photoactivation only within target cells and to cause a bioactive substance to express bioactivity only within the specific target cells.

### 2. Preparation of the Caged Compound of the Present Invention

The caged compound of the present invention, K-Q-X, can be prepared by binding a photocleavable protecting group to a bioactive substance to prepare compound Q-X, and then binding a compound, which can be an enzyme substrate, to the compound. The caged compound of the present invention can also be prepared by binding a compound, which can be an enzyme substrate, to a photocleavable protecting group to prepare compound K-Q, and then binding a bioactive substance to the compound.

Binding of a bioactive substance to a photocleavable protecting group; that is, the protection of a bioactive substance with a photocleavable protecting group can be performed by methods described in known documents. In addition, the protection of a bioactive substance with a photocleavable protecting group may be performed for a functional group such that the bioactive substance loses bioactivity or exhibits suppressed bioactivity as a result of protection.

For example, the complexes of photocleavable protecting groups and various bioactive substances can be synthesized, as described in the following documents.
1. T. Furuta, et al., Proc. Natl. Acad. Sci. U.S.A., 96, 1193-1200 (1999)
2. H. Ando et al., Nature Genetics, 28, 317-325 (2001).
3. A. Z. Suzuki et al., Org. Lett., 5, 4867-4870 (2003).
4. T. Nishigaki et al., Dev. Biol. 272, 376-388 (2004).
5. T. Furuta et al., ChemBioChem, 5, 1119-1128 (2004).
6. H. Ando et al., Methods in Cell Biology, 77, 159-171 (2004).
7. C. D. Wood et al., J Cell Biol. 169, 725-731 (2005)
8. H. Ando et al., Dev Biol; 287, 456-468 (2005)
9. T. Furuta et al., Org. Lett. 9, 4717-4720 (2007)
10. T. Kawakami et al., ChemBioChem, 9, 1583-1586 (2008)
11. K. Katayama et al., Chem Commun, 5399-5401 (2008).
12. E. J. Quann et al., Nature Immun. 10, 627-635 (2009).
13. S. Yamaguchi et al., Chem. Commun. 46, 2244-2246, (2010)
14. S. Mizukami et al., J. Am. Chem. Soc. 132, 9524-9525 (2010)
15. W. Nomura et al., ChemBioChem, 12, 535-539 (2011)
16. T. Furuta, Journal of Synthetic Organic Chemistry, Japan, 69, 1164-1169 (2012)
17. T. Furuta et al., Org. Lett. 14, 6182-6185 (2012).
18. T. Furuta et al., Trends Anal. Chem. 23, 501-509 (2004).
19. T. Furuta et al., Chapter 1.2: Coumarin-4-ylmethyl Phototriggers, in “Dynamic Studies in Biology: Phototriggers, Photoswitches and Caged Biomolecules,” M. Goeldner and R. S. Givens Eds, WILEY-VCH, pp 29-55 (2005)
20. WO00/31588
21. WO2006/093083
22. US2002/0155606
23. JP Patent Publication (Kokai) No. 2002-315576 A

Of these documents, document 15 describes a method for synthesizing Bhc-various bioactive substances, document 10 describes a method for synthesizing Bhc-peptide, documents 2, 9, 17, 18 and 21 describe methods for synthesizing Bhc-nucleic acid, document 5 describes a method for synthesizing Bhc-nucleoside, document 1 describes a method for synthesizing Bhc-glutamic acid, and document 3 describes a method for synthesizing a compound having a Bhc-OH group.

Based on these descriptions, a caged compound represented by Q-X can be synthesized by allowing various bioactive substances to be bound to Bhc groups or Bhcmoc group. Similarly, a caged compound represented by Q-X can be synthesized by allowing the xanthene-type photocleavable protecting group or the resorufin-type photocleavable protecting group to be bound to a bioactive substance.

### Synthesis of Xanthene-Type Photocleavable Protecting Group

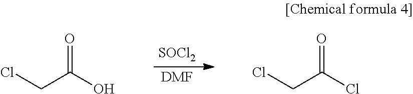

Chloroacetic acid (1.9095 g, 20.2 mmol) and thionyl chloride (1.5 mL, 21.21 mmol) were added to a 10-mL eggplant flask, and then a drop of DMF was added. A condenser and a calcium chloride tube were attached to the flask, and then the solution was stirred while heated at 90° C. for 3 hours. The reaction solution was allowed to cool to a room temperature, and then the solvent was removed under reduced pressure.

¹H NMR (DMSO-d6) δ 4.27 (s, 2H)

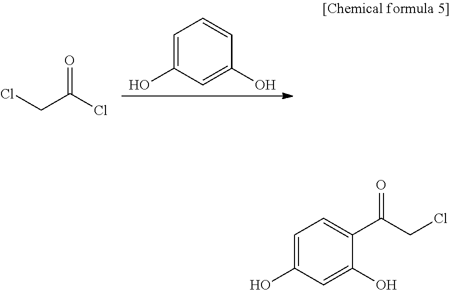 [SMILES: O=C(Cl)CCl ; Oc1cccc(O)c1 ; O=C(CCl)c1ccc(O)cc1O]

Aluminum chloride (2.0039 g, 1.4 mmol) was added to a 20-mL eggplant flask, and then nitrobenzene (2.5 mL) and chloroacetyl chloride (0.08 mL, 2 mmol) were added. Resorcinol (0.055 g, 0.5 mmol) was added to the flask while stirring at 0° C. The reaction solution was allowed to cool to room temperature, and then stirred for 18 hours. After separation with chloroform, the organic layer was dried with magnesium sulfate and then filtered. The solvent was removed under reduced pressure. The thus obtained product was purified by flash column chromatography (elution solvent: hexane/ethyl acetate=4/1), and thus 0.51 g (2.73 mmol, 52% yield) of a target product, 2-chloro-1-(2,4-dihydroxyphenyl)ethanone, was obtained.

¹H NMR (DMSO-d6) δ 5.01 (s, 2H), 6.33 (d, 1H, J=2.4 Hz), 6.39 (dd, 1H, J=2.4 & 8.7 Hz), 7.73 (d, 1H, J=8.7 Hz)

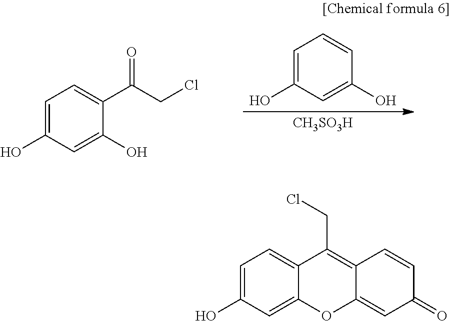

A condenser and a calcium chloride tube were attached to a 30-mL eggplant flask. 2-chloro-1-(2,4-dihydroxyphenyl)ethanone (0.119 g, 0.64 mmol) was added, and then resorcinol (0.07 g, 0.67 mmol) and methane sulfonic acid (0.5 mL) were added to the flask. After stirring at 90° C. for 1 hour, the reaction solution was allowed to cool to room temperature, and then a precipitate generated by adding water was collected by suction filtration. The precipitate was dried under a vacuum, and thus 0.11 g (0.41 mmol, 64% yield) of a target product, 9-(6-hydroxy-3-oxo-3H-xanthen-9-yl)chloromethyl, was obtained.

¹H NMR (DMSO-d6) δ 6.53 (d, 1H, J=2.4 Hz), 6.58 (d, 1H, J=2.4 Hz), 6.61 (s, 2H), 6.65 (d, 1H, J=2.4 Hz), 6.68 (d, 1H, J=2.4 Hz), 7.51 (d, 1H, J=9 Hz), 8.23 (d, 1H, J=9 Hz)¹³C NMR (DMSO-d6) δ 102.47, 102.70, 105.73, 110.62, 111.13, 112.42, 112.74, 124.96, 125.82, 128.68, 150.81, 152.65, 158.82, 159.05

Compound K, which can be an enzyme substrate, may be bound to a Bhc group that is a photocleavable protecting group or a phenolic OH group that is a xanthene-type or resorufin-type photocleavable protecting group via the functional group of K. K can be bound to the photocleavable protecting group by a known method.

The present invention further encompasses a compound represented by K-Q or Q-X that is a precursor compound for the synthesis of a caged compound represented by K-Q-X. For example, a compound represented by K-Q is prepared by binding photocleavable protecting group Q to compound K (that can be an enzyme substrate), which is a precursor compound for the synthesis of a caged compound, wherein a group for binding to K has been bound to Q. An example of the precursor compound is a precursor compound prepared by binding a diazo group (⁻N═N⁺—) to Q for binding to a nucleic acid such as DNA, cDNA, mRNA, siRNA, miRNA, and antisense RNA, or a nucleotide such as ATP, ADP, cAMP, GTP, GDP, GTP-γ-A, GDP-(β-S, cGMP, 8-bromo cAMP, 8-chloro cAMP, 8-bromo cGMP, 8-chloro cGMP, 8-para-chlorophenylthio cAMP, and 8-para-chlorophenylthio cGMP. Specific examples of a precursor compound prepared by binding K to 6-bromo-4-diazomethyl-7-hydroxycoumarin include Gal-Bhc-diazo and AcGal-Bhc-diazo. The precursor compound can be converted to a caged compound represented by K-Q-X by binding X that is a nucleic acid or the like through a reaction between a phosphoric acid group of a nucleic acid or the like and a diazo group.

### 3. Use of the Caged Compound of the Present Invention

The caged compound of the present invention represented by K-Q-X is used as follows.

(1) An enzyme that is expressed in target cells, into which the caged compound of the present invention containing a bioactive substance is introduced so as to cause the bioactive substance to act, is determined. At this time, an enzyme that is specifically expressed in target cells is preferably selected.

When an exogenous enzyme is used in this case, the enzyme is introduced into target cells by the above method.

(2) As substrate K of the caged compound of the present invention K-Q-X, a substrate for the selected enzyme is selected. In addition, a bioactive substance to be caused to express bioactivity in target cells is selected. As a photocleavable protecting group, any one of the above examples may be used.

(3) K-Q-X is synthesized.

(4) K-Q-X is introduced into target cells. K-Q-X can be introduced into target cells using a known drug delivery system, for example. For example, K-Q-X is encapsulated in a microcapsule such as a liposome, and then a substance binding to the surfaces of target cells is bound to the microcapsule, and thus K-Q-X can be introduced into target cells.

(5) When K-Q-X is introduced into target cells, compound K, which can be an enzyme substrate, is cleaved by an endogenous enzyme within the target cells or an enzyme that is expressed within cells after introduction via transformation, resulting in bioactive substance Q-X being protected by a photocleavable protecting group. Subsequently, cells are irradiated with light having a specific wavelength, so that the photocleavable protecting group is photoactivated, and Q is dissociated from K.

A photocleavable protecting group can be photoactivated by the irradiation of a compound represented by Q-X with excitation light having a specific wavelength. The wavelength of the excitation light should be a wavelength such that the excitation light is not absorbed by cellular endogenous molecules and is desirably 300 nm or greater (λmax). When a photocleavable protecting group is a Bhc group or a Bhcmoc group, irradiation is performed with light having a wavelength ranging from 400 nm to 450 nm, and preferably a wavelength of approximately 405±10 nm. When a photocleavable protecting group is a xanthene-type photocleavablelytic protecting group or a resorufin-type photocleavable protecting group, the irradiation is performed with light having a wavelength ranging from 450 nm to 600 nm. The time for the irradiation ranges from 0.1 to 5.0 seconds, preferably ranges from 0.5 to 2.0 seconds, and further preferably ranges from 0.5 to 1.0 seconds. Photoirradiation is performed for cells into which the caged compound of the present invention has been introduced. In vivo cells can be irradiated with light from outside the body. Furthermore, a photoirradiation means is set at the tip of a catheter. This photoirradiation means is caused to reach a position where in vivo target cells are present via blood vessels and the like, and then the position may be irradiated with light.

(6) As a result, K expresses bioactivity within cells.

### EXAMPLES

Hereafter, the present invention is described in greater detail with reference to the following examples, although the present invention is not limited to the examples.

### Example 1

### Examination of the Photoreactivity of Bhcmoc-Propargyl and Gal-Bhcmoc-Propargyl

### 1. Synthesis of Gal-Bhcmoc-Propargyl

Bhcmoc-propargyl (the compound represented by the rightmost formula of the following chemical formula 7) was synthesized from 6-bromo-7-hydroxy-4-hydroxymethylcoumarin-(BhcCH₂—OH). Propargyl amine generated after photoirradiation is a model compound of a bioactive molecule having an amino group.

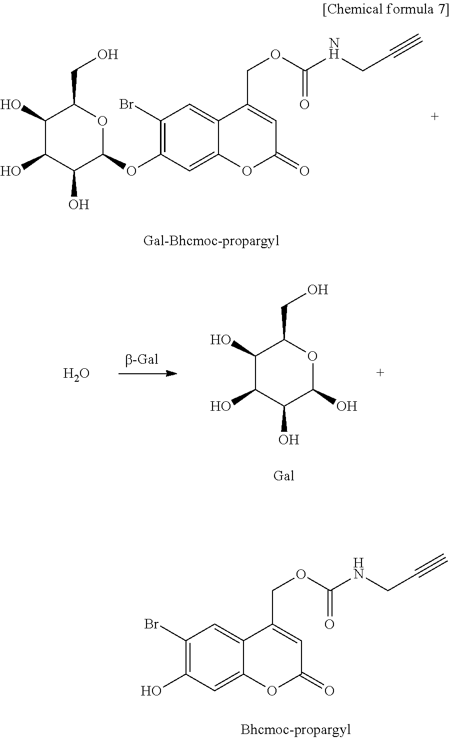 [SMILES: C#CCNC(=O)OCc1cc(=O)oc2cc(O[C@@H]3O[C@H](CO)[C@H](O)[C@H](O)[C@@H]3O)c(Br)cc12 ; O ; OC[C@H]1O[C@@H](O)[C@@H](O)[C@@H](O)[C@H]1O ; C#CCNC(=O)OCc1cc(=O)oc2cc(O)c(Br)cc12]

The synthesis method was as follows.

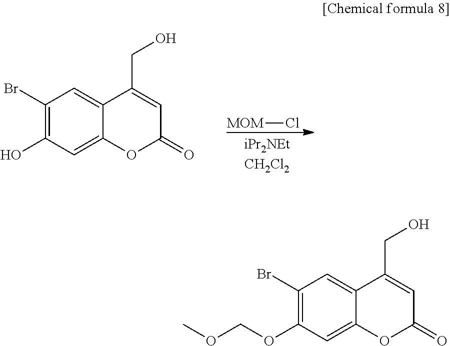

Bhc-CH₂OH (1.3546 g, 5.00 mmol), dehydrated dichloromethane (15 mL), iPr₂NEt (1.2 mL, 6.9 mmol), and MOM-Cl (494 mL, 6.05 mmol) were added to a 50-mL eggplant flask, and then the solution was stirred at room temperature for 2 hours under an argon atmosphere. After separation with 0.5 M aqueous citric acid solution and chloroform, the organic layer was dried with magnesium sulfate. The solvent was removed under reduced pressure, a solution of hexane/chloroform=1/1 was added for suspension, and then the precipitate was collected by suction filtration. The precipitate was dried under a vacuum, and thus 1.2433 g (3.95 mmol, 79% yield) of a target product, 6-bromo-7-methoxymethoxy-4-hydroxymethylcoumarin, was obtained. ¹H NMR (CDCl₃) δ·3.52 (3H, s), 4.86 (2H, dd, J=6 & 1.5 Hz), 5.31 (2H, s), 6.52 (1H, t, J=1.5 Hz), 7.16 (1H, s), 7.70 (1H, s)

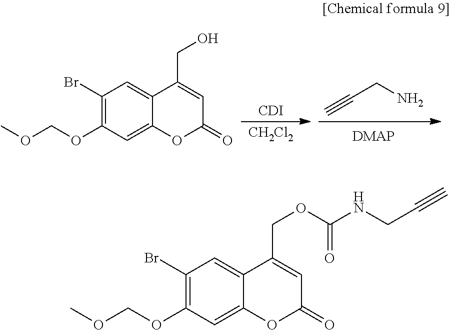

6-bromo-7-methoxymethoxy-4-hydroxymethylcoumarin (315.4 mg, 1.00 mmol) and dehydrated dichloromethane (2 mL) were added to a 10-mL eggplant flask, a calcium chloride tube was attached to the flask, and then the solution was stirred at 0° C. When CDI (196.2 mg, 1.21 mmol) was added to the solution, the solution was cloudy and a spinner remained unrotated. Hence, dehydrated dichloromethane (2 mL) was further added, followed by 1.5 hours of stirring at room temperature. The reaction progress was confirmed with TLC (developing solvent: hexane/ethyl acetate=1/1), indicating that the raw materials still seemed to remain. A further reaction was performed for 1 hour with the addition of CDI (24.7 mg, 0.152 mmol). DMAP (160.5 mg, 1.31 mmol) and propargyl amine (85.6 μL, 1.35 mmol) were added, followed by 1.5 hours of stirring. After the solvent had been removed under reduced pressure, dichloromethane was added for suspension, and thus the precipitate was obtained by suction filtration. The precipitate was dried under a vacuum, and then 218.9 mg (0.552 mmol, 55% yield) of a target product, (6-bromo-7-methoxymethoxycoumarin-4-yl)methoxycarbonyl propargylamide, was obtained.

¹H NMR (DMSO-d₆) δ·3.15 (1H, t, J=2.1 Hz), 3.42 (3H, s), 3.84 (2H, dd, J=2.1 & 5.4 Hz), 5.32 (2H, s), 5.43 (2H, s), 6.28 (1H, s), 7.28 (1H, s), 7.98 (1H, s), 8.03 (1H, t, J=5.4 Hz)

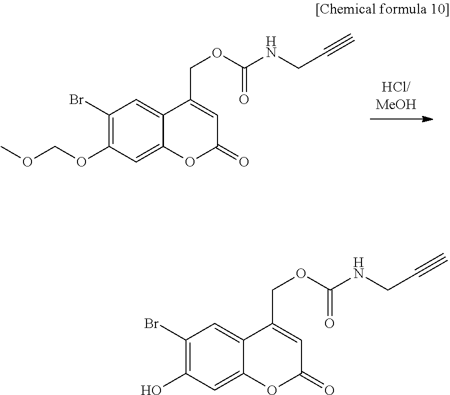 [SMILES: C#CCNC(=O)OCc1cc(=O)oc2cc(OCOC)c(Br)cc12 ; C#CCNC(=O)OCc1cc(=O)oc2cc(O)c(Br)cc12]

(6-bromo-7-methoxymethoxycoumarin-4-yl)methoxycarbonyl propargylamide (99.9 mg, 0.252 mmol) and HCl/methanol (2 mL (HCl: 2.5 mmol)) were added to a 10-mL eggplant flask, followed by 24 hours of stirring at room temperature. The reaction progress was confirmed with TLC (developing solvent: dichloromethane/methanol=20/1), indicating that the raw materials seemed to remain. Hence, HCl/methanol (0.5 mL (HCl: 0.6 mmol)) was added for further 3 hours of reaction. The solvent was removed under reduced pressure, dried under a vacuum, and thus 88.5 mg (0.251 mmol, 100% yield) of a target product, (6-bromo-7-hydroxycoumarin-4-yl)methoxycarbonylpropargylamide (Bhcmoc-propargyl), was obtained.

¹H NMR (DMSO-d₆) δ·3.15 (1H, t, J=2.1 Hz), 3.84 (2H, dd, J=5.7 & 2.1 Hz), 5.30 (2H, s), 6.18 (1H, s), 6.92 (1H, s), 7.88 (1H, s), 8.02 (1H, t, J=5.7 Hz)

Furthermore, a galactosyl group was bound to Bhcmoc-propargyl with the following reaction formula, thereby synthesizing Gal-Bhcmoc-propargyl.

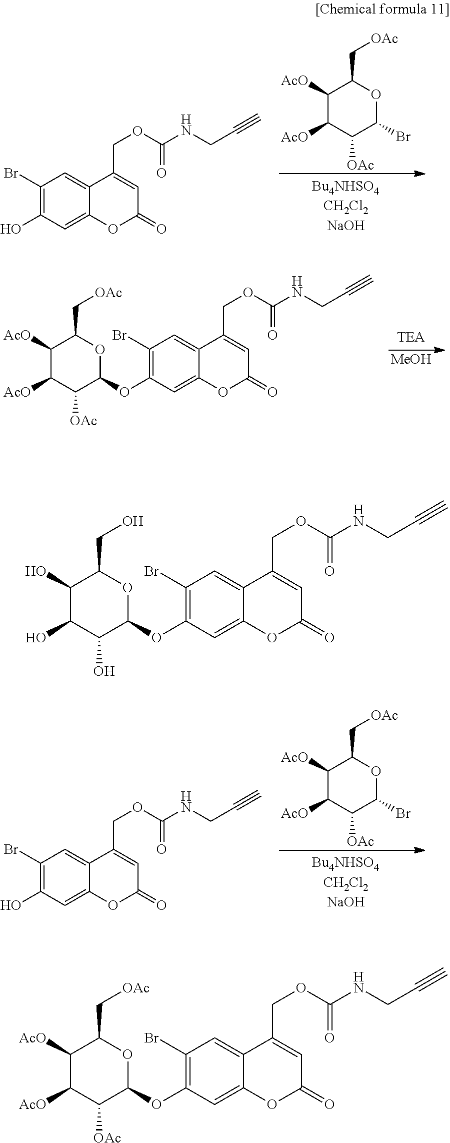

Bhcmoc-propargyl (69.9 mg, 0.199 mmol), 2,3,4,6-tetra-O-acetyl-α-D-galactopyranosyl bromide (92.0 mg, 0.224 mmol), Bu₄NHSO₄ (81.8 mg, 0.241 mmol), dichloromethane (4 mL), and 1 M NaOH (600 μL, 0.600 mmol) were added to a 20-mL eggplant flask, followed by 4 hours of stirring at room temperature. After separation, the organic layer was collected in another flask. The solvent was then removed under reduced pressure. The resultant was purified by flash column chromatography (elution solvent: dichloromethane/methanol=100/1), and thus 64.0 mg (93.7 μmol, 47% yield) of a target product, AcGal-Bhcmoc-propargyl, was obtained.

¹H NMR (CDCl₃) δ·2.03 (3H, s), 2.11 (3H, s), 2.15 (3H, s), 2.21 (3H, s), 2.31 (1H, t, J=2.1 Hz), 4.04 (2H, dd, J=2.1 & 5.4 Hz), 4.19-4.25 (3H, m), 5.13-5.19 (2H, m), 5.26 (1H, d, J=4.5 Hz), 5.50 (2H, d, J=3.3 Hz), 5.60-5.66 (1H, m), 6.41 (1H, s), 7.19 (1H, s), 7.70 (1H, s)

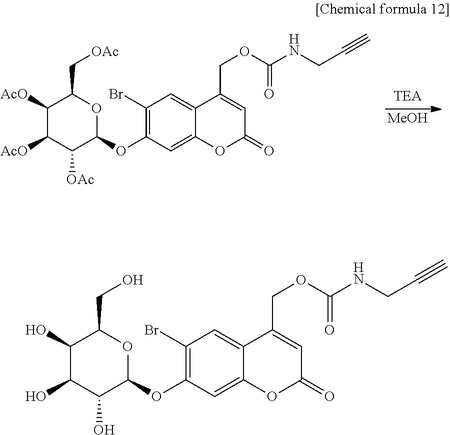 [SMILES: "MeOH" ; C#CCNC(=O)OCc1cc(=O)oc2cc(O[C@@H]3O[C@H](COC(C)=O)[C@H](OC(C)=O)[C@H](OC(C)=O)[C@H]3OC(C)=O)c(Br)cc12 ; C#CCNC(=O)OCc1cc(=O)oc2cc(O[C@@H]3O[C@H](CO)[C@H](O)[C@H](O)[C@H]3O)c(Br)cc12]

Ac-GalBhcmoc-propargyl (50.0 mg, 73.3 μmol), TEA (14.6 μL, 0.105 mmol), and methanol (5 mL) were added to a 20-mL eggplant flask, followed by 14 hours of stirring at 65° C. The reaction progress was confirmed with TLC (dichloromethane/methanol=10/1), indicating that the raw materials disappeared, but some Ac groups seemed to remain undissociated. Hence, TEA (4.86 μL, 34.9 μmol) was added, followed by a further 1 hour of stirring at 65° C. The solvent was removed under reduced pressure, the resultant was washed with chloroform, and then the product was obtained by suction filtration. After drying under a vacuum, 14.7 mg (28.6 μmol, 39% yield) of a target product, Gal-Bhcmoc-propargyl, was obtained.

¹H NMR (DMSO-d₆) δ·3.16 (1H, s), 3.41-3.56 (3H, m), 3.63-3.72 (3H, m), 3.84 (2H, d, J=3.0 Hz), 5.15 (1H, d, J=6.0 Hz), 5.34 (2H, s), 6.63 (1H, s), 7.31 (1H, s), 7.97 (1H, s), 8.05 (1H, t, J=6.0 Hz)

¹³C NMR (DMSO-d₆) δ 29.84, 48.48, 60.19, 61.08, 67.93, 69.84, 73.26, 75.71, 80.94, 100.48, 103.57, 107.14, 109.60, 112.01, 128.23, 150.69, 153.56, 155.03, 155.98, 159.48

### 2. Examination of the Photoreactivity of Bhcmoc-Propargyl and Gal-Bhcmoc-Propargyl

A 10⁻⁵ M KMOPS solution (0.1% DMSO, pH 7.3) of Gal-Bhcmoc-propargyl was prepared, and then analyzed using HPLC. As a result, one peak was detected at 325 nm or 350 nm. We determined that it could be used for photocleaving experiments with no problem.

### (1) UV Spectrum

UV spectra were measured for the 10⁻⁵ M KMOPS solution (0.1% DMSO, pH 7.3) of Gal-Bhcmoc-propargyl and the 10⁻⁵ M KMOPS solution (0.1% DMSO, pH 7.3) of Bhcmoc-propargyl.

FIG. 7 shows UV spectra and table 1 shows absorption wavelengths.

TABLE 1

| | Gal-Bhcmoc-propargyl | Bhcmoc-propargyl |
|---|---|---|
| λₘₐₓ(nm) | 286, 326 | 369 |
| εₘₐₓ (M⁻¹ cm⁻¹) | 7000, 9600 | 17400 |

### (2) Photocleavings

The 10⁻⁵ M KMOPS solution (0.1% DMSO, pH 7.3) of Gal-Bhcmoc-propargyl or Bhcmoc-propargyl was irradiated with light with 405 nm. The reaction progress was monitored over photoirradiation time by C18 reverse phase HPLC. Table 2 shows HPLC conditions.

TABLE 2

| | Gal-Bhcmoc-propargyl | Bhcmoc-propargyl |
|---|---|---|
| Column | Nakalai COSMOSIL packed column | |
| | 5C18-AR-II (4.6 mm × 250 mm) | |
| Elution | 15-45% CH₃CN in H₂O (10 min) | 50% CH₃CN in H₂O |
| conditions | | |

| | |
|---|---|
| Flow rate | 0.8 mL/min |
| Detection | 325 nm |
| wavelength | |

A peak of Gal-Bhcmoc-propargyl was observed at around the retention time (RT) of 9.5 min. Even when the photoirradiation time was gradually increased in order of 0 second, 5 seconds, 10 seconds, 15 seconds, 20 seconds, 30 seconds, 60 seconds, and 300 seconds, no decrease was confirmed for Gal-Bhcmoc-propargyl.

A peak of Bhcmoc-propargyl was observed at around the retention time (RT) of 6.0 min and a peak of a photocleavaed product (thought to be Bhc-OH) was observed at around the same of 4.4 min. When the photoirradiation time was increased in order of 0 second, 5 seconds, 10 seconds, 15 seconds, 20 seconds, and 30 seconds, decreases in Bhcmoc-propargyl and increases in the photocleaved product could be confirmed.

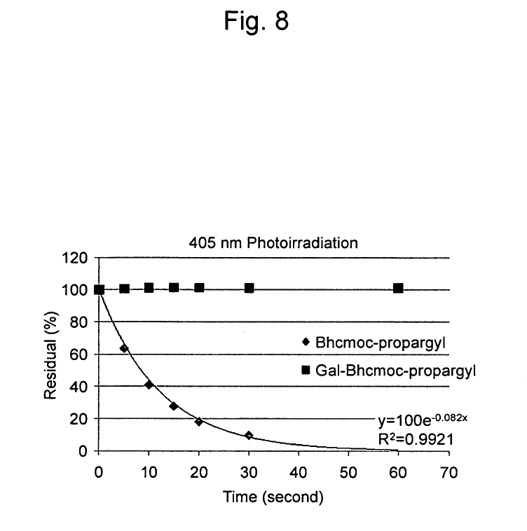

Decreases in Bhcmoc-propargyl and Gal-Bhcmoc-propargyl as a result of photocleaving are shown in the graph (FIG. 8). It was revealed that Gal-Bhcmoc-propargyl was not photocleaved, but Bhcmoc-propargyl was photocleaved by 405-nm photoirradiation.

### 3. Confirmation of the Conversion of Gal-Bhcmoc-Propargyl to Bhcmoc-Propargyl in the Presence of β-Gal

To 0.1 M phosphate buffer (pH 7.3) containing Gal-Bhcmoc-propargyl (final concentration of 15 μM), mercaptoethanol (0.11 M), and magnesium chloride (1 mM), β-Gal was added to a final concentration of 0.25 ng/μL. While the reaction solution was kept at 37° C., the reaction progress was followed by HPLC (see Table 2 for analytical conditions). It was confirmed that Gal-Bhcmoc-propargyl was completely converted to Bhcmoc-propargyl after 1 hour.

The results of this Example demonstrated that photocleaving did not take place in the absence of β-Gal, but photocleaving took place in the presence of β-Gal by 405-nm photoirradiation.

The results of this Example demonstrated that: (1) a Gal-Bhc group can be synthesized; (2) the absorption maximum is present near 330 nm (see FIG. 7); (3) a synthesized Gal-Bhc group serves as a substrate of β-Gal in an aqueous solution (pH 7), and then quantitatively converted to a Bhc group; (4) a Gal-Bhc caged compound does not react at all when irradiated with light with 405 nm; and (5) the Bhc caged compound after reaction with β-Gal can be uncaged with high efficiency by 405-nm photoirradiation.

### Example 2

### Synthesis of Caged Fluorescein with AcGal Introduced Therein

### (1) Synthesis of Dipropargyl Fluorescein

Dipropargyl fluorescein was synthesized with the following reaction formula.

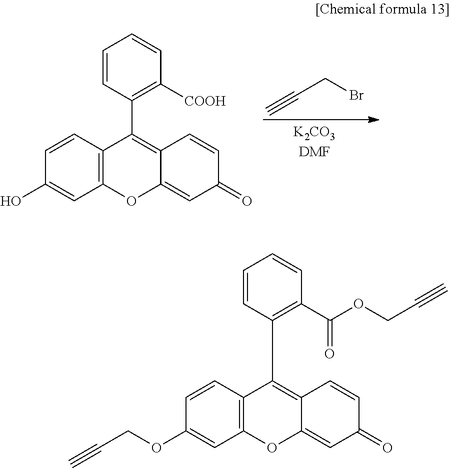

Fluorescein (3.32 g, 9.99 mmol), K₂CO₃ (3.87 g, 27.6 mmol), propargyl bromide (3.5 mL, 39.3 mmol), and dehydrated DMF (20 mL) were added to a 100-mL eggplant flask, followed by 4 hours of stirring at 65° C. under an argon atmosphere. The solvent was removed under reduced pressure, the resultant was suspended with H₂O, and then the precipitate was obtained by suction filtration. As a result of NMR confirmation, some peaks indicating those other than the target product were observed. Hence, purification was attempted by the next reaction. The amount of the thus obtained sample was 4.12 g (10.1 mmol; quant).

¹H NMR (CDCl₃) δ 2.33 (1H, t, J=2.4 Hz), 2.61 (1H, t, J=2.4 Hz), 4.60 (2H, dd, J=4.2 & 2.7 Hz), 4.80 (2H, d, J=2.4 Hz), 6.46 (1H, d, J=2.0 Hz), 6.55 (1H, dd, J=10.2 & 2.0 Hz), 6.78-6.92 (3H, m), 7.06 (1H, d, J=2.4 Hz), 7.33 (1H, d, J=7.5 Hz), 7.66-7.99 (2H, m), 8.28 (1H, d, J=7.5 Hz)

### (2) Synthesis of Propargyl Fluorescein

Propargyl fluorescein was synthesized with the following reaction formula.

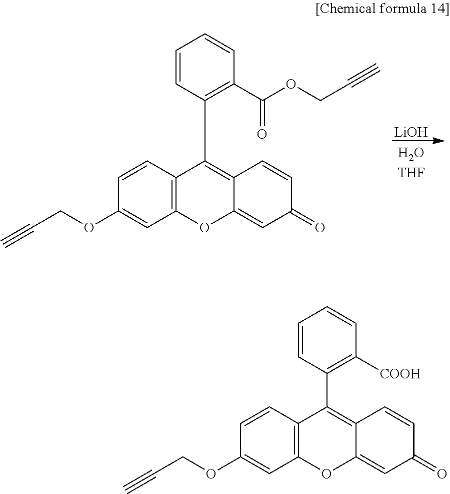

Dipropargyl fluorescein (0.818 g, 2.00 mmol), LiOH.H₂O (0.545 g, 13.0 mmol), H₂O (6.7 mL), and THF (6.8 mL) were added to a 50-mL eggplant flask, followed by 6 hours of stirring at room temperature. Since the raw materials remained undisappeared, LiOH.H₂O (41.4 mg, 0.987 mmol) was added and the solution was further stirred for 17 hours. THF alone was removed under reduced pressure, HCl was added to quench the reaction, and then chloroform was added for separation. The organic layer was dried with MgSO₄, and then the solvent was removed under reduced pressure. The thus obtained product was purified by flash column chromatography (elution solvent: CH₂Cl₂/MeOH=40/1), so that 155.9 mg (0.421 mmol, 21.0% yield) of a target product was obtained.

¹H NMR (CDCl₃) δ 2.56 (1H, t, J=2.3 Hz), 4.72 (2H, d, J=2.4 Hz), 5.30 (1H, s), 6.53 (1H, dd, J=8.7 and 2.3 Hz), 6.64-6.75 (3H, m), 6.87 (1H, d, J=2.1 Hz), 7.16 (1H, d, J=7.2 Hz), 7.59-7.70 (2H, m), 8.02 (1H, d, J=7.2 Hz)

### (3) Synthesis of MOMBhcmoc-Propargyl Fluorescein

MOMBhcmoc-propargyl fluorescein was synthesized with the following reaction formula.

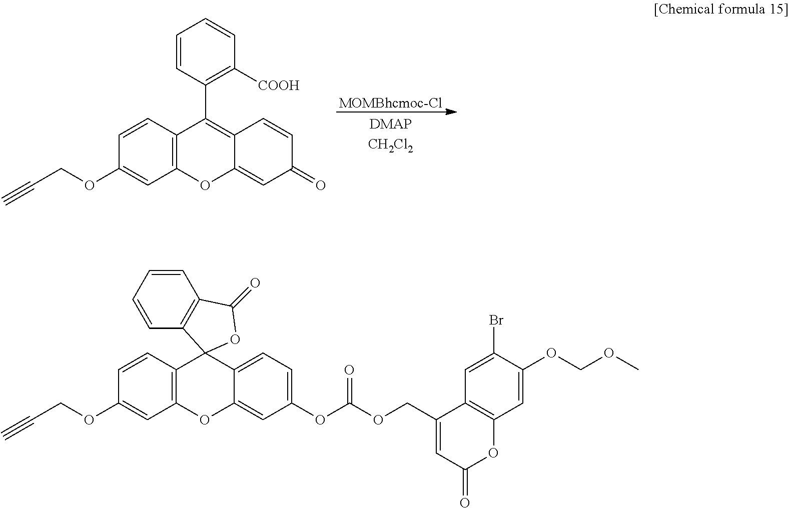 [SMILES: C#CCOc1ccc2c(-c3ccccc3C(=O)O)c3ccc(=O)cc-3oc2c1 ; "MOMBhcmoc-Cl" ; C#CCOc1ccc2c(c1)Oc1cc(OC(=O)OCc3cc(=O)oc4cc(OCOC)c(Br)cc34)ccc1C21OC(=O)c2ccccc21]

Propargyl fluorescein (106.1 mg, 0.287 mmol), MOMBhcmoc-Cl (203.6 mg, 0.539 mmol), DMAP (78.1 mg, 0.639 mmol), and dehydrated CH₂Cl₂ (10 mL) were added to a 50-mL eggplant flask, followed by 22.5 hours of stirring at room temperature under an argon atmosphere. HCl was added to quench the reaction, and then chloroform was added for separation. The organic layer was dried with MgSO₄, and then the solvent was removed under reduced pressure. Purification of the thus obtained product was attempted by flash column chromatography (elution solvent: CH₂Cl₂/MeOH=150/1). The amount of the thus obtained sample was 185.9 mg (0.261 mmol, 90.9% yield). The thus obtained sample was subjected to deprotection of MOM.

¹H NMR (CDCl₃) δ 2.60 (1H, t, J=1.8 Hz), 3.52 (3H, s), 4.73 (2H, d, J=1.8 Hz), 5.32 (2H, s), 6.45 (2H, d, J=15.9 Hz), 6.70-6.75 (2H, m), 6.83-6.95 (3H, m), 7.14-7.21 (3H, m), 7.62-7.72 (4H, m), 8.03 (1H, d, J=7.2 Hz)

### (4) Synthesis of Bhcmoc-Propargyl Fluorescein

Bhcmoc-propargyl fluorescein was synthesized with the following reaction formula.

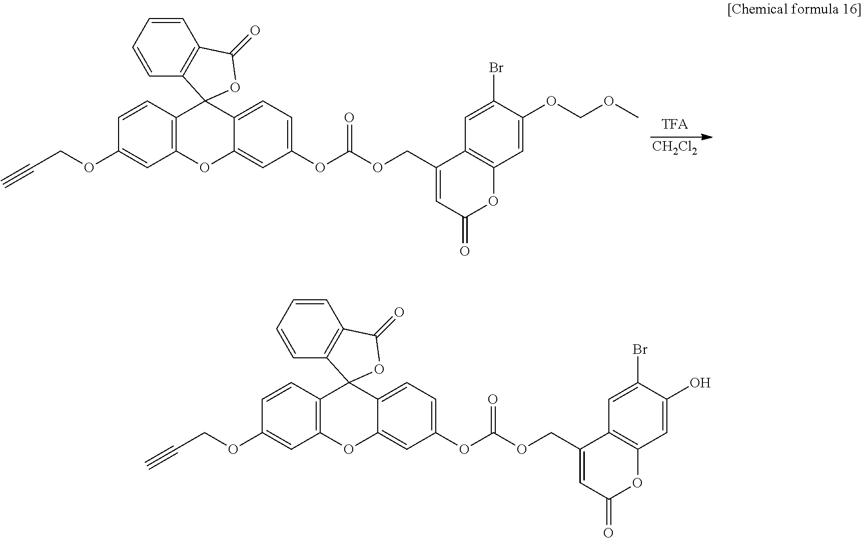

To examine the conditions for deprotection, deprotection was started with a small amount of the sample.

MOMBhcmoc-propargyl fluorescein (53.8 mg, 0.0756 mmol), dehydrated CH₂Cl₂ (3 mL), and TFA (1 mL) were added to a 10-mL eggplant flask, followed by 2.5 hours of stirring at room temperature. The solvent was removed under reduced pressure, and then NMR was confirmed (*). Deprotection was confirmed, and then the remaining MOMBhcmoc-propargyl fluorescein was similarly subjected to a deprotection reaction.

MOMBhcmoc-propargyl fluorescein (132.1 mg, 0.186 mmol), dehydrated CH₂Cl₂ (3 mL), and TFA (1 mL) were added to a 30-mL eggplant flask, followed by 2 hours of stirring at room temperature. As a result of NMR confirmation, the result was almost the same as that obtained above (*). Hence, the all amount of the sample was purified. The thus obtained product was filtered with CH₂Cl₂, the filtrate was purified by flash column chromatography (elution solvent: CH₂Cl₂/MeOH=80/1), and thus 89.8 mg (0.135 mmol, 51.6% yield) of a target product was obtained.

¹H NMR (CDCl₃) δ 2.57 (1H, t, J=2.1 Hz), 4.72 (2H, d, J=2.1 Hz), 5.38 (2H, s), 6.44 (1H, s), 6.71 (2H, s), 6.82-6.94 (5H, m), 7.02 (1H, s), 7.14-7.20 (2H, m), 7.62-7.72 (3H, m), 8.04 (1H, d, J=7.5 Hz)

### (5) Synthesis of AcGal-Bhcmoc-Propargyl Fluorescein

AcGal-Bhcmoc-propargyl fluorescein was synthesized with the following reaction formula.

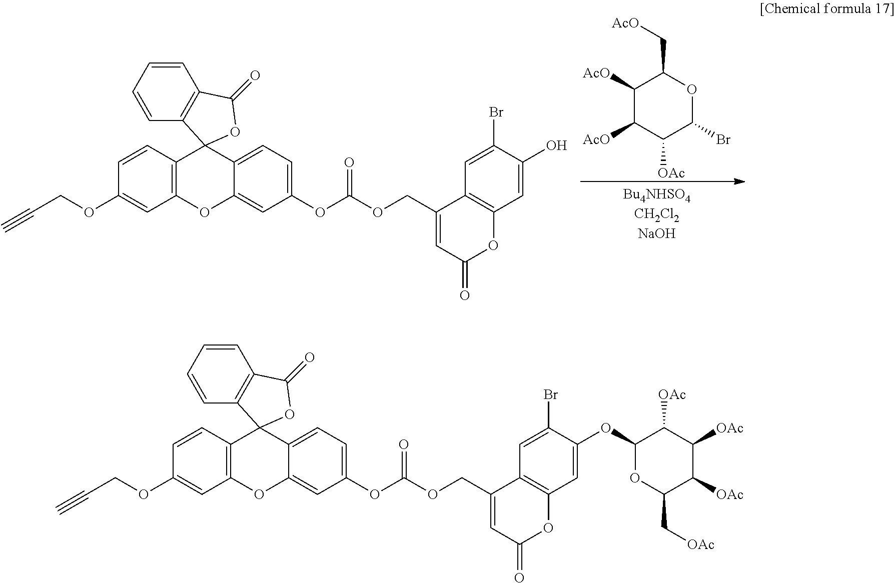 [SMILES: C#CCOc1ccc2c(c1)Oc1cc(OC(=O)OCc3cc(=O)oc4cc(O)c(Br)cc34)ccc1C21OC(=O)c2ccccc21 ; CC(=O)OC[C@H]1O[C@H](Br)[C@H](OC(C)=O)[C@@H](OC(C)=O)[C@H]1OC(C)=O ; C#CCOc1ccc2c(c1)Oc1cc(OC(=O)OCc3cc(=O)oc4cc(O[C@@H]5O[C@H](COC(C)=O)[C@H](OC(C)=O)[C@H](OC(C)=O)[C@H]5OC(C)=O)c(Br)cc34)ccc1C21OC(=O)c2ccccc21]

Bhcmoc-propargyl fluorescein (49.8 mg, 0.0746 mmol), Acetobromo-α-D-galactose (33.6 mg, 0.0817 mmol), Bu₄NHSO₄ (30.9 mg, 0.0910 mmol), CH₂Cl₂ (1.5 mL), and 1N NaOH (225 μL, 0.225 mmol) were added to a 10-mL eggplant flask, followed by 5.5 hours of stirring at room temperature. The organic layer was collected, the solvent was removed under reduced pressure, the thus obtained product was purified by flash column chromatography (elution solvent: CH₂Cl₂/MeOH=50/1), and thus 19.1 mg (0.0191 mmol, 25.6% yield) of a target product was obtained.

¹H NMR (CDCl₃) δ 2.02 (3H, s), 2.06 (3H, s), 2.12 (3H, s), 2.16 (3H, s), 2.58 (1H, t, J=2.1 Hz), 4.04-4.24 (5H, m), 4.73 (1H, d, J=2.1 Hz), 5.03-5.11 (2H, m), 5.50-5.53 (2H, m), 6.54 (1H, s), 6.70-6.76 (3H, m), 6.82-6.94 (3H, m), 7.17-7.22 (3H, m), 7.62-7.74 (3H, m), 8.04 (1H, d, J=7.2 Hz)

(6) Synthesis of 15-chloro-3,6,9-trioxapentadecyl azide

15-chloro-3,6,9-trioxapentadecyl azide was synthesized with the following reaction formula.

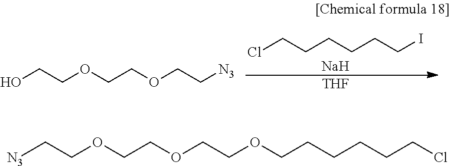

NaH (54.7 mg, 2.28 mmol), 2-(2-(2-azidoethoxy)ethoxy)ethanol (160.3 mg, 0.915 mmol), and THF (3 mL) were added to a 30-mL eggplant flask, followed by 2 hours of stirring at room temperature under an argon atmosphere. 1-chloro-6-iodohexane (152 μL, 1.01 mmol) was added to the solution, followed by 3.5 hours of stirring. Since the raw materials remained on TLC, 1-chloro-6-iodohexane (28 μL, 0.183 mmol) was added and then the solution was further stirred for 16 hours. Since the raw materials still remained, NaH (11.2 mg, 0.467 mmol) was added and then the solution was stirred for 5.5 hours. However, TLC remained unchanged. H₂O was added to quench the reaction, and then the solvent was removed under reduced pressure. CHCl₃ and 1M HCl were added to the flask and then the solution was stirred. The organic layer was collected and dried with MgSO₄, and then the solvent was removed under reduced pressure. The thus obtained product was purified by flash column chromatography (elution solvent: hexane/EtOAc=6/1), and thus 42.6 mg (0.145 mmol, 15.8% yield) of a target product was obtained.

(7) Click Reaction of AcGal-Bhcmoc-Propargyl Fluorescein and 15-chloro-3,6,9-trioxapentadecyl azide

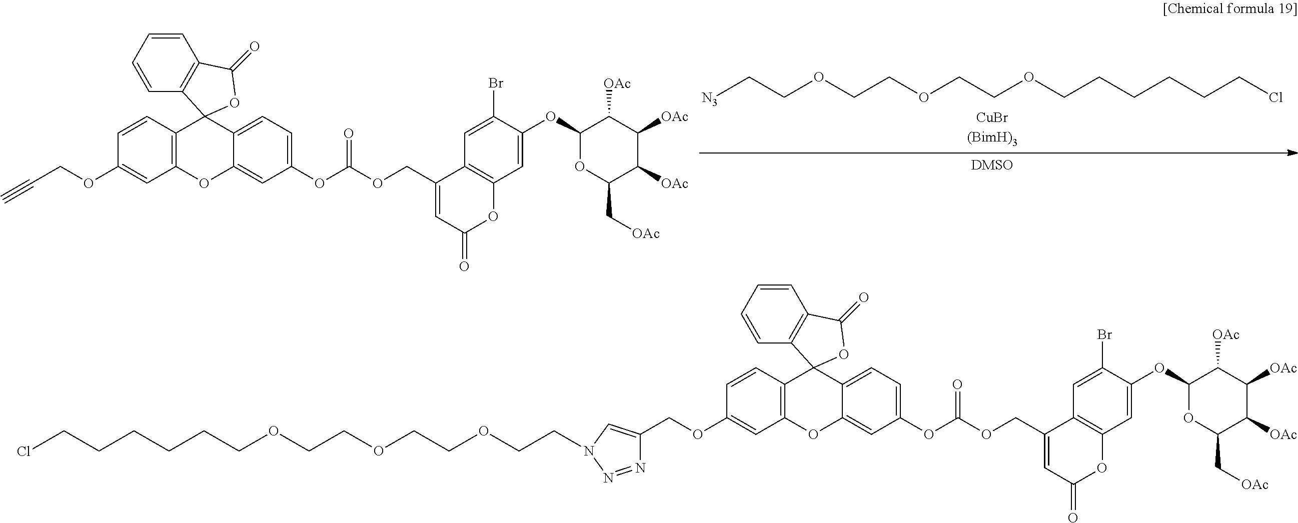 [SMILES: C#CCOc1ccc2c(c1)Oc1cc(OC(=O)OCc3cc(=O)oc4cc(O[C@@H]5O[C@H](COC(C)=O)[C@H](OC(C)=O)[C@H](OC(C)=O)[C@H]5OC(C)=O)c(Br)cc34)ccc1C21OC(=O)c2ccccc21 ; [N-]=[N+]=NCCOCCOCCOCCCCCCCl ; CC(=O)OC[C@H]1O[C@@H](Oc2cc3oc(=O)cc(COC(=O)Oc4ccc5c(c4)Oc4cc(OCc6cn(CCOCCOCCOCCCCCCCl)nn6)ccc4C54OC(=O)c5ccccc54)c3cc2Br)[C@H](OC(C)=O)[C@@H](OC(C)=O)[C@H]1OC(C)=O]

AcGal-Bhcmoc-propargyl fluorescein (9.5 mg, 0.0095 mmol), 15-chloro-3,6,9-trioxapentadecyl azide (4.9 mg, 0.017 mmol), (BimH)₃ (4.2 mg, 0.010 mmol), and CuBr (7.3 mg, 0.051 mmol) were added to a 10-mL eggplant flask, followed by 2.5 hours of stirring at room temperature under an argon atmosphere. The solvent was removed under reduced pressure. NMR confirmation demonstrated that the target product was contained.

### Example 3

### Method for Synthesizing AcGal-Bhc-cAMP

### (1) Synthesis of Bhc-CH₂OTBDMS

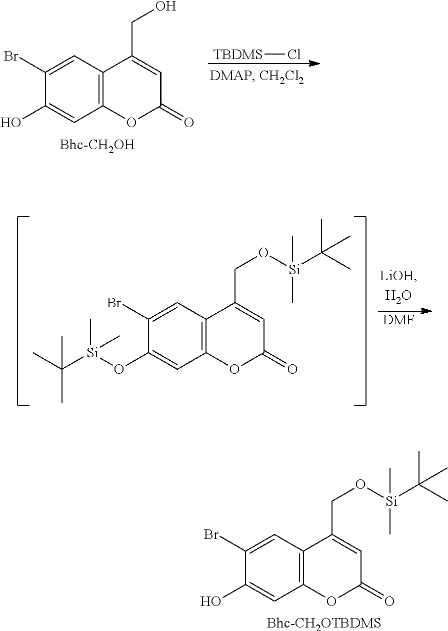

Bhc-CH₂OH (1361.7 mg, 5.0236 mmol), tert-butyldimethylsilyl chloride (2237.0 mg, 14.842 mmol), and DMAP (2109.6 mg, 17.268 mmol) were dissolved in dichloromethane (30 mL), and then the solution was stirred at room temperature for 30 minutes. The reaction was stopped with a 0.5 M aqueous citric acid solution, the organic layer was extracted twice with dichloromethane, and then the organic layer was concentrated to dryness under reduced pressure. The thus obtained partially purified product was dissolved in DMF (14 mL), lithium hydroxide monohydrate (627.3 mg, 14.950 mmol) was added, and then the resultant was stirred at room temperature for 30 minutes. The reaction was stopped with a 0.5 M aqueous citric acid solution, and then extraction was performed 3 times with chloroform. The organic layer was dried with magnesium sulfate, and then concentrated to dryness. The crude product was suspended in a mixed solvent of chloroform/hexane=1/2. The precipitate was collected and then dried under a vacuum, and thus a target compound, Bhc-CH₂OTBDMS (1612.3 mg, 4.1842 mmol, 83%) was obtained.

¹H NMR (300 MHz, CDCl₃) δ 7.60 (s, 1H), 7.02 (s, 1H), 6.50 (s, 1H), 4.80 (s, 1H), 0.96 (s, 6H), 0.16 (s, 9H)

### (2) Synthesis of AcGal-Bhc-CH₂OTBDMS

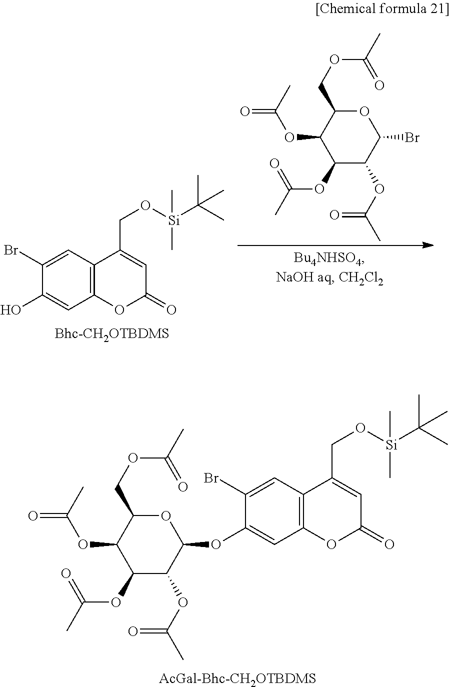

Acetobromo-α-D-galactose (1230.6 mg, 2.9927 mmol), tetrabutylammonium hydrogen sulfate (1083.9 mg, 3.1924 mmol), dichloromethane (30 mL), 1 M aqueous sodium hydroxide solution (8 mL), and Bhc-CH₂OTBDMS (771.9 mg, 2.003 mmol) were mixed, and then the mixture was vigorously stirred at room temperature for 15 hours. The organic layer was extracted with chloroform, dried with magnesium sulfate, and then concentrated under reduced pressure. The thus obtained partially purified product was purified by flash column chromatography (silica gel, elution solvent: dichloromethane/methanol=50/1). Thus, compound 4 (334.3 mg, 0.4671 mmol, 23%) and a partially purified product (639.4 mg); that is the mixture of a target compound, AcGal-Bhc-CH₂OTBDMS, and acetobromo-α-D-galactose were obtained.

¹H NMR (300 MHz, CDCl₃) δ 7.63 (s, 1H), 7.16 (s, 1H), 6.54 (s, 1H), 5.63 (dd, 1H, J=8.1 Hz, 11.0 Hz), 5.49 (br, 1H), 5.14 (dd, 1H J=3.3 Hz, 11.0 Hz), 5.16 (d, 1H, J=8.1 Hz), 4.80 (s, 2H), 4.22 (d, 2H, J=5.7 Hz), 4.16-4.12 (m, 1H), 2.20 (s, 3H), 2.16 (s, 3H), 2.11 (s, 3H), 2.03 (s, 3H) 0.96 (s, 9H), 0.16 (s, 6H)

### (3) Synthesis of AcGal-Bhc-CH₂OH

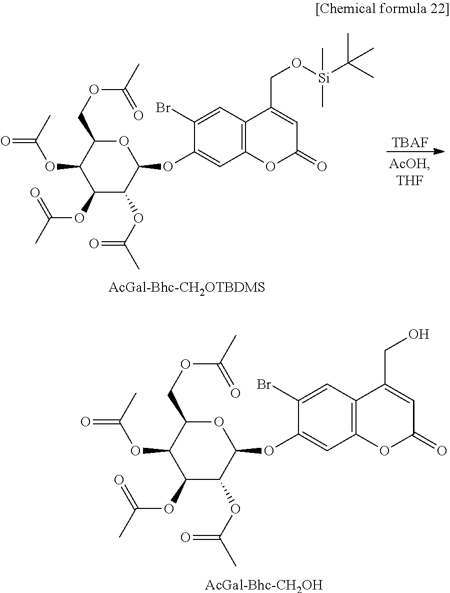

A 1M tetrabtylammonium fluoride THF solution (200 μL) and acetic acid (13.74 μL) were dissolved in THF (3.3 mL) and then the solution was mixed well. AcGal-Bhc-CH₂OTBDMS (171.6 mg, 0.2854 mmol) was added to the solution, followed by 20 minutes of stirring at room temperature. The reaction was stopped with a 0.5 M aqueous citric acid solution, and THF alone was distilled off under reduced pressure. The aqueous layer was extracted twice with chloroform, followed by concentration under reduced pressure. The thus obtained partially purified product was suspended in a mixed solvent of chloroform/hexane=1/2. The precipitate was collected and then dried under a vacuum, so that a white powdery target compound, AcGal-Bhc-CH₂OH (109.2 mg, 0.1816 mmol, 64%), was obtained. ¹H NMR (300 MHz, CDCl₃) δ 7.70 (s, 1H), 7.33 (s, 1H), 6.56 (s, 1H), 5.63 (dd, 1H, J=8.1 Hz, 11.0 Hz), 5.50 (br, 1H), 5.14 (dd, 1H J=3.3 Hz, 11.0 Hz), 5.15 (d, 1H, J=8.1 Hz), 4.86 (s, 2H), 4.23 (d, 2H, J=5.7 Hz), 4.16-4.12 (m, 1H), 2.21 (s, 3H), 2.17 (s, 3H), 2.11 (s, 3H), 2.03 (s, 3H)

### (4) Synthesis of AcGal-Bhc-CHO

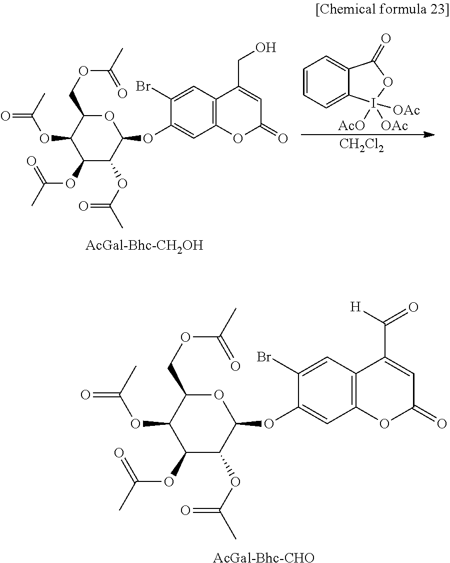

AcGal-Bhc-CH₂OH (30.31 mg, 0.05040 mmol) was dissolved in dichloromethane (1.5 mL), and then Dess-Martin periodinane (23.37 mg, 0.05510 mmol) was added, followed by 2.5 hours of stirring at room temperature. Dess-Martin periodinane (4.81 mg, 0.01134 mmol) was added, and then the solution was further stirred for 1.5 hours at room temperature. The reaction solution was purified by flash column chromatography (silica gel, elution solvent: dichloromethane/methanol=60/1), and thus a light yellow solid target compound, AcGal-Bhc-CHO (29.5 mg, 0.04922 mmol, 98%), was obtained.

¹H NMR (300 MHz, CDCl₃) δ 10.05 (s, 1H), 8.85 (s, 1H), 7.20 (s, 1H), 6.82 (s, 1H), 5.63 (dd, 1H, J=8.1 Hz, 11.0 Hz), 5.51 (br, 1H), 5.17-5.08 (m, 2H), 4.28-4.14 (m, 3H), 2.21 (s, 3H), 2.17 (s, 3H), 2.11 (s, 3H), 2.03 (s, 3H)

### (5) Synthesis of AcGal-Bhc-diazo

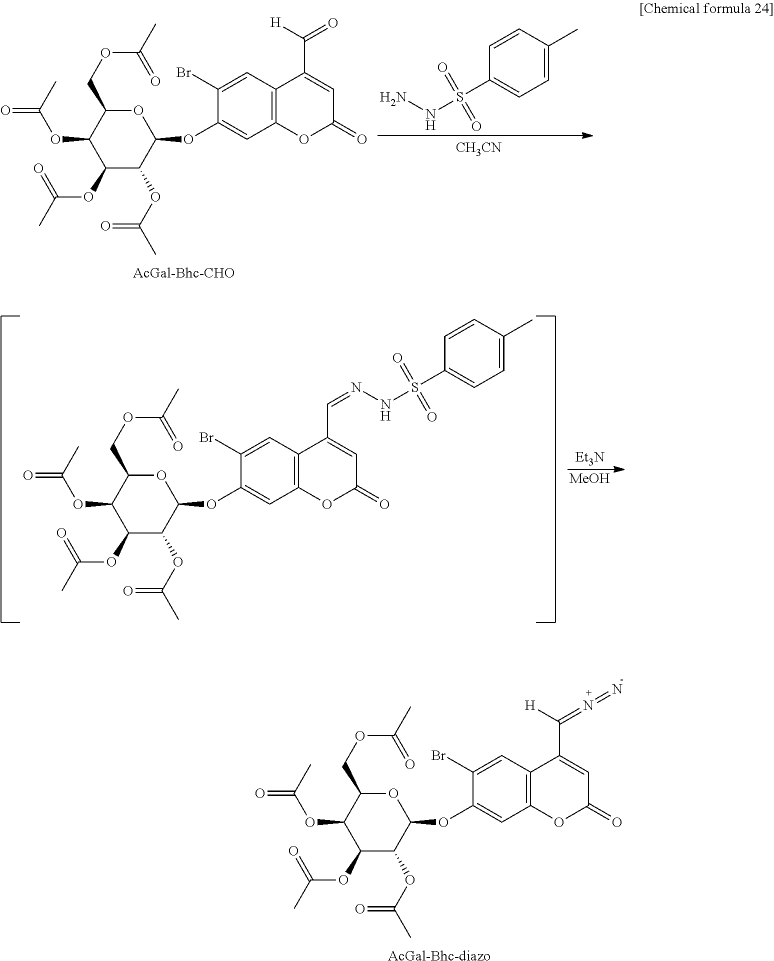

AcGal-Bhc-CHO (306.6 mg, 0.5116 mmol) and p-toluenesulfonyl hydrazide (106.61 mg, 0.5725 mmol) were dissolved in acetonitrile (9 mL), followed by 2 hours and 15 minutes of stirring at 45° C. The solvent was distilled off under reduced pressure, and then the resultant was purified by flash column chromatography (silica gel, elution solvent: dichloromethane/methanol=40/1 to 30/1), so that a mixture of diazo compound 1 and hydrazone was obtained. Methanol (10 mL) and triethylamine (30.3 μL) were added to the mixture, followed by 2 hours of stirring at 0° C. The precipitate was collected and then dried under a vacuum, so that a light yellow powdery target compound, AcGal-Bhc-diazo (206.84 mg, 0.3128 mmol, 62%), was obtained.

¹H NMR (300 MHz, CDCl₃) δ 7.49 (s, 1H), 7.20 (s, 1H), 7.15 (s, 1H), 5.80 (s, 1H), 5.64 (dd, 1H, J=8.1 Hz, 11.0 Hz), 5.50 (br, 1H), 5.25 (s, 1H), 5.14 (dd, 1H J=3.3 Hz, 11.0 Hz), 5.07 (d, 1H, J=8.1 Hz), 4.22 (d, 2H, J=5.7 Hz), 4.16-4.12 (m, 1H), 2.20 (s, 3H), 2.16 (s, 3H), 2.11 (s, 3H), 2.03 (s, 3H)

### (6) Synthesis of AcGal-Bhc-cAMP

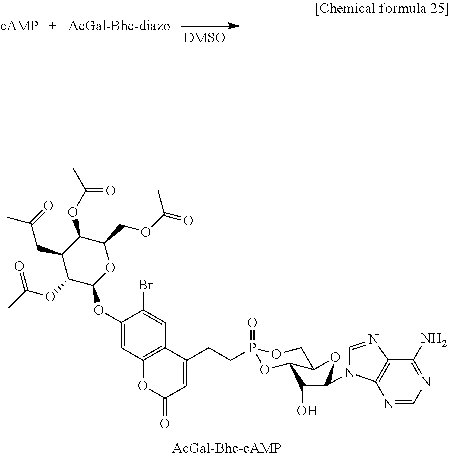

AcGal-Bhc-diazo (28.4 mg, 0.0467 mmol), cAMP (50.3 mg, 0.153 mmol), and DMSO (1 mL) were added to a 10-mL eggplant flask, followed by 5 hours of stirring at 50° C. under an argon atmosphere. The resultant was left to cool to room temperature, and then the solvent was distilled off under reduced pressure. The thus obtained crude product was purified by flash column chromatography (10 g of silica gel 60, elution solvent: dichloromethane/methanol=20/1), so that 21.7 mg (0.0238 mmol, 51% yield) of target AcGal-Bhc-cAMP was obtained. FIG. 6D shows the structural formula of AcGal-Bhc-cAMP.

¹H NMR (300 MHz, DMSO-d₆) Isomeric mixture of an isomer with an AcGal-Bhc group bound via axial linkage and an isomer with the same bound via equatorial linkage. Data in parentheses indicate the chemical shifts of minor isomers. δ 8.39 (8.35) (s, 1H), 8.19 (8.13) (1H, s), 8.09 (8.04) (1H, s), 7.31 (7.33) (1H, s), 6.49 (6.60) (1H, s), 6.35 (6.41) (1H, d, J=5 Hz), 6.09 (6.07) (1H, s), 5.64 (5.67) (1H, s), 5.51-5.25 (6H, m), 4.80-4.18 (8H, m), 2.17 (3H, s), 2.07 (2.08) (3H, s), 2.06 (2.05) (3H, s), 1.97 (3H, s).

### INDUSTRIAL APPLICABILITY

The caged compound of the present invention, which is represented by K-Q-X, is characterized by masking of the activity of a bioactive substance using two keys. The bioactive substance can express activity when the two keys are opened in arbitrary target cells within an arbitrary period of time. Thus the caged compound can be used for diagnostic, therapeutic, and research applications.

1. A compound represented by the following general formula (I), in which bioactive substance X, photocleavable protecting group Q, and compound K, which can be an enzyme substrate and is dissociated from Q-X by an enzyme reaction are bound.
[Chemical formula 1]
K-Q-X (I)

2. The compound according to claim 1 represented by the following general formula (I), in which the bioactive substance X, the photocleavable protecting group Q, and the compound K, which can be an enzyme substrate and is dissociated from Q-X by an enzyme reaction are bound, wherein
Q is a protecting group that is photocleaved by light with a specific wavelength and then dissociated from X, when K is not bound thereto;
X is a substance that does not express bioactivity when Q is bound thereto, but expresses bioactivity when Q is dissociated therefrom; and
K is dissociated from Q by the enzyme so as to generate a compound represented by Q-X, and K expresses bioactivity when the compound represented by Q-X is photoirradiated to cause the dissociation of Q from X.
[Chemical formula 2]
K-Q-X (I)

3. The compound according to claim 1, wherein the enzyme for causing the dissociation of the compound K, which can be an enzyme substrate, from Q-X, is an intracellular endogenous enzyme or an exogenous enzyme that is introduced into cells, and X expresses bioactivity within cells.

4. The compound according to claim 1, wherein the photocleavable protecting group Q is selected from the group consisting of a Bhc group, a Bhcmoc group, a xanthene-type photoclaevable protecting group, and a resorufin-type photocleavable protecting group.

5. The compound according to claim 1, wherein a combination of the compound that can be an enzyme substrate and the enzyme is selected from the group consisting of a combination of galactose and β-galactosidase, a combination of glucose and glucosidase, a combination of glucuronic acid and glucuronidase, and a combination of phosphoric acid and alkaline phosphatase.

6. The compound according to claim 1, wherein the bioactive substance X is selected from the group consisting of a protein, a nucleic acid, a fatty acid, and an amino acid.

7. The compound according to claim 1, wherein the bioactive substance X is selected from the group consisting of an enzyme inhibitor, a hormone, a lipid-signal molecule, a neurotransmitter, a microtubule depolymerization inhibitor, a microtubule polymerization inhibitor, an antitumor antibiotic, a topoisomerase inhibitor, a purine metabolism inhibitor, a ribonucleotide reductase inhibitor, a pyrimidine metabolism inhibitor, an antifolate, and an alkylating agent.

8. The compound according to claim 1, wherein the photocleavable protecting group Q is a Bhc group, the compound K, which is an enzyme substrate and is dissociated from Q-X by an enzyme reaction, is galactose, and galactose is dissociated from Q-X by β-galactosidase.

9. A method for causing X to express bioactivity within cells, comprising the steps of: introducing the compound K-Q-X of claim 1, into cells; causing the dissociation from Q-X of the compound K, which can be an enzyme substrate and is dissociated from Q-X by an enzyme reaction within cells; and causing the dissociation of Q from Q-X by photoirradiation.

10. The method for causing X to express bioactivity within cells according to claim 9, wherein the enzyme is a cell endogenous enzyme.

11. The method for causing X to express bioactivity within cells according to claim 9, wherein the enzyme is an exogenous enzyme.

12. The method for causing X to express bioactivity within cells according to claim 9, wherein the photocleavable protecting group Q is a Bhc group or a Bhcmoc group, and photoirradiation is performed with a wavelength ranging from 400 nm to 450 nm.

13. The method for causing X to express bioactivity within cells according to claim 9, wherein the compound K, which is an enzyme substrate and is dissociated from Q-X by an enzyme reaction, is galactose, and galactose is dissociated from Q-X by β-galactosidase.

14. A precursor compound of the compound represented by K-Q-X according to claim 1, which:
is represented by K-Q, wherein the photocleavable protecting group Q and the compound K, which can be an enzyme substrate and is dissociated from Q-X by an enzyme reaction, are bound to each other; and
is converted to K-Q-X by binding the bioactive substance X thereto.

15. The precursor compound according to claim 14, wherein:
the bioactive substance X that can be bound to the precursor compound is a compound selected from the group consisting of DNA, cDNA, mRNA, siRNA, miRNA, antisense RNA, ATP, ADP, cAMP, GTP, GDP, GTP-γ-A, GDP-β-S, cGMP, 8-bromo cAMP, 8-chloro cAMP, 8-bromo cGMP, 8-chloro cGMP, 8-para-chlorophenylthio cAMP, and 8-para-chlorophenylthio cGMP;
the photocleavable protecting group Q is a Bhc group or a Bhcmoc group to which a diazo group is bound; and
the compound K, which can be an enzyme substrate, is galactose.
